(19) United States
(12) Patent Application Publication
(10) Pub. No.: US 2023/0346716 A1
(43) Pub. Date: Nov. 2, 2023

(54) TREATMENT OF SKIN DISORDERS WITH TOPICAL TAPINAROF COMPOSITIONS

(71) Applicant: Sol-Gel Technologies Ltd., Ness Ziona (IL)

(72) Inventors: Moshe ARKIN, Kfar Shmaryahu (IL); Danil Finkel-Moiseev, Rehovot (IL); Hila Hakak Djerbi, Tel Aviv (IL); Karine Neimann, Ness Ziona (IL)

(73) Assignee: Sol-Gel Technologies Ltd., Ness Ziona (IL)

(21) Appl. No.: 18/350,513

(22) Filed: Jul. 11, 2023

Related U.S. Application Data
(63) Continuation-in-part of application No. 17/425,956, filed on Jul. 27, 2021.
(60) Provisional application No. 62/797,298, filed on Jan. 27, 2019.
(60) Provisional application No. 63/359,936, filed on Jul. 11, 2022.

(51) Int. Cl. A61K 31/05; A61K 9/06; A61K 47/44; A61K 47/10; A61K 47/18; A61K 47/12; A61P 17/06; A61K 47/26; A61K 47/14; A61K 47/06; A61K 47/32; A61K 47/20

(52) U.S. Cl. CPC A61K 31/05; A61K 9/06; A61K 47/44; A61K 47/10; A61K 47/183; A61K 47/12; A61P 17/06; A61K 47/26; A61K 47/14; A61K 47/06; A61K 47/32; A61K 47/20

(57) ABSTRACT

Provided herein are topical composition comprising tapinarof and combination compositions comprising a therapeutically effective dose of tapinarof and a therapeutically effective dose of at least one additional active agent, selected from at least one corticosteroid, calcipotriene, at least one JAK inhibitor and combinations thereof, and a carrier suitable for topical administration.

The above compositions are useful for the treatment, prevention or alleviation of skin disorders selected from psoriasis and atopic dermatitis and exhibit synergistic or additive effects which allow reducing the doses of the active agents in the compositions.

### CROSS-REFERENCE TO RELATED APPLICATIONS

This Application is a Continuation-in-Part of U.S. patent application Ser. No. 17/425,956 filed Jul. 27, 2021 which is a National Phase Application of PCT International Application No. PCT/IL2020/050103, International Filing Date Jan. 27, 2020, claiming the benefit of U.S. Patent Application No. 62/797,298, filed Jan. 27, 2019; and this Application claiming the benefit of U.S. Patent Application No. 63/359,936, filed Jul. 11, 2022, which are incorporated in their entirety herein by reference.

### FIELD OF THE INVENTION

The present invention, in some embodiments thereof, relates to treatment of skin disorders by topical administration of a composition comprising tapinarof and combination compositions comprising tapinarof and at least one additional active agent. The compositions of this invention are useful for the treatment, prevention or amelioration of skin disorders and exhibit synergistic or additive effects which allow reducing the dose of the active agents in the combination composition.

### BACKGROUND OF THE INVENTION

Tapinarof (3,5-dihydroxy-4-isopropyl-trans-stilbene) is a pharmaceutical active agent investigated for the treatment of atopic dermatitis, psoriasis and psoriatic disorders (Zang Y N, et al., Int J Clin Pharmacol Ther. 2016 February; 54(2):87-95). The 3,5-dihydroxy-4-isopropyl-stilbene is also known as benvitimod.

Tapinarof is a first-in-class drug, whose mechanism is not yet fully understood.

Tapinarof is being developed by Glaxo Smith Kline (Stiefel, a GSK company) and Dermavant as a topical drug for treatment of mild to moderate plaque psoriasis and atopic dermatitis. It was shown in both mouse models and in vitro human skin studies to inhibit specific proinflammatory mediators, including interleukin-6 and interleukin-17A, and enhance skin barrier function (J Invest Dermatol. 2017 October; 137[10]:2110-9).

A number of GSK28994512 (tapinarof) clinical studies were completed:
1. Dose finding study (Phase I) of GSK28994512 in subjects with plaque psoriasis-1% (10 mg/g) and 0.5% (5 mg/g) creams for two application frequencies, once a day and twice a day, vs. vehicle cream.
2. Dose finding study of GSK28994512 (Phase II) in subjects with atopic dermatitis—1% (10 mg/g) and 0.5% (5 mg/g) creams for two application frequencies, once a day and twice a day, vs. vehicle cream.
3. Dose finding study (Phase II) of GSK28994512 in subjects with plaque psoriasis—1% (10 mg/g) and 0.5% (5 mg/g) creams for two application frequencies, once a day and twice a day, vs. vehicle cream.
4. Skin irritation study (Phase I) of GSK28994512 cream (0.5% and 1%) vs vehicle cream.
5. A single dose (Phase I) exploratory study in healthy volunteers with GSK28994512 cream for atopic dermatitis. Two cream formulations (GSK2894512 cream A and GSK2894512 cream B) were tested in combination for skin residency.
6. Pharmacokinetic study (Phase I) of topical GSK2894512 1% and 2% creams following twice daily application of the 2% cream (cohort 1) or 1% cream (cohort 2).

According to Jancin B. (Dermatology News, Nov. 11, 2017), in the above studies, the 1% tapinarof arm showed higher efficacy and had a quicker onset of action than the 0.5% arm or vehicle. The most frequent adverse events associated with tapinarof were folliculitis and contact dermatitis.

Tapinarof, which seems to be a significant advance in psoriasis treatment, presents however higher adverse effects (44.5%) when compared to placebo (20.2%) and calcipotriene (19.5%) as a single drug (Medscape Mar. 5, 2017 report by Frellick M., on Abstr. 5629, Amer. Acad. of Dermatology meeting, Mar. 4, 2017).

Psoriasis is an autoimmune disease, characterized by typically red, scaly patches of skin. The following main types of psoriasis include: plaque, guttate, inverse, pustular, flexural/inverse and erythrodermic. Plaque psoriasis (psoriasis vulgaris) is the most common form of psoriasis.

Inverse psoriasis is a rare form of psoriasis which is also known as flexural or intertriginous psoriasis. This subtype of psoriasis can occur in any area where two skin surfaces meet. Classically the skin of the groin region, armpits and genitals are affected. In these regions the skin appears red, shiny, and moist, with clear borders, and can sometimes crack in the centre.

Some of the known topical psoriasis treatments use pharmaceutical active agents like corticosteroids like desoxymethasone and Vitamin D analogues like calcipotriene or paricalcitol. Combinations of several of the above classes of active agents, like Vitamin D and corticosteroids have been investigated. Most of the known psoriasis topical treatments in general, and those comprising steroids in particular, present undesirable side-effects.

Though a number of psoriasis treatments are available, most treatments bring about symptom alleviation or remission rather than complete cure.

There is an unmet need for methods for the treatment of skin disorders using tapinarof topical compositions, devoid of serious side-effects.

### BRIEF DESCRIPTION OF THE DRAWINGS

The subject matter regarded as the invention is particularly pointed out and distinctly claimed in the concluding portion of the specification. This invention, however, both as to organization and method of operation, together with objects, features, and advantages thereof, may best be understood by reference to the following detailed description when read with the accompanying drawings in which:

FIG. 1 depicts epidermal thickness in human xenograft psoriasis model in SCID mice following treatment with the tapinarof cream or dexamethasone vs. vehicle.

FIG. 2 depicts epidermal thickness in human xenograft psoriasis model in SCID mice following treatment with the tapinarof cream A or cream D vs dexamethasone.

FIG. 3 depicts proliferation index in human xenograft psoriasis model in SCID mice following treatment with the tapinarof cream A or cream D vs dexamethasone.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### SUMMARY OF THE INVENTION

This invention provides a topical composition comprising from about 0.25% w/w to about 2.0% w/w tapinarof and at least one additional active agent, selected from about 0.01% w/w to about 0.25% w/w at least one corticosteroid of potency class 1 or 2 or from about 0.0025% w/w to about 2.5% w/w at least one corticosteroid of potency class 3-7, from about 0.001% w/w to about 0.005% w/w calcipotriene, from about 0.1% w/w to about 3% w/w at least one JAK1, JAK2 or JAK 1/2 inhibitor and combinations thereof, and a carrier suitable for topical administration.

The composition is suitable for the treatment, prevention or amelioration of skin disorders selected from psoriasis and atopic dermatitis, and exhibits synergistic or additive effects which allow reducing the dose of the active agents in the combination compositions.

In some embodiments provided herein a topical cream composition, comprising from about 1% w/w to about 2% w/w tapinarof or a pharmaceutically acceptable salt thereof and less than 1% w/w surfactant. In another embodiment, the surfactant is ionic or non-ionic. In another embodiment, the surfactant is anionic.

In some embodiments, provided herein a topical cream composition, comprising from about 0.25% w/w to about 10.0% w/w tapinarof or a pharmaceutically acceptable salt thereof, from about 0.1% w/w to about 20% surfactant, and optionally from about 0.01% to about 1% w/w an anti-oxidant, wherein the surfactant is ionic. In another embodiment, the surfactant is anionic.

In another embodiment, provided herein a method of treating a skin disorder in a patient in need thereof, said method comprises administering to said patient the topical cream composition provided herein, wherein said skin disorder is selected from the group consisting from psoriasis, toxic and allergic contact eczema, atopic eczema, seborrheic eczema, lichen simplex, sunburn, pruritus (e.g. in the genitoanal region), alopecia areata, hypertrophic scars, discoid lupus erythematosus, follicular and extensive pyodermas, acne, rosacea, atopic dermatitis, prurigo nodularis and hidradenitis suppurativa.

### DETAILED DESCRIPTION OF THE INVENTION

The present invention provides novel methods of treatment and topical compositions useful for the treatment, prevention and amelioration and of skin disorders selected from psoriasis and atopic dermatitis.

In some embodiment, the present invention provides a topical compositions comprising tapinarof and at least one additional active agent selected from at least one corticosteroid, Vitamin D analogue, at least one JAK inhibitor and combinations thereof, wherein the combination composition exhibits improved therapeutic effects and also synergistic or additive effects in the treatment of skin disorders selected from psoriasis and atopic dermatitis and, as a result, allow using lower dosage of the actives and alleviate the side-effects (like local irritation and contact dermatitis).

In another embodiment the Vitamin D analogue is calcipotriene, paricalcitol or hydrate thereof. In another embodiment, the Vitamin D analogue is calcipotriene or hydrate thereof. In another embodiment, the Vitamin D analogue is paricalcitol or hydrate thereof.

The tapinarof combination compositions of this invention have a double advantage vs. the use of tapinarof as a single drug: on the one hand the at least one additional active has the potential to alleviate the tapinarof side-effects and on the other hand the synergistic or additive effect enables using lower active agent dosages.

### Corticosteroids

The at least one corticosteroid is selected from steroids of various potencies (see below Class 1-Class 7), approved and marketed in the US for topical use.

### Topical Steroid Classification by Potency

According to the “Topical steroid potency chart” of the National Psoriasis Foundation (NPF), the various marketed topical drugs comprising steroids belong to the following potency classes, according to the steroid and the topical drug strength. Due to the different topical drug strength, drugs of different strengths and/or different dosage forms may belong to more than one steroid class. The percentages in parentheses are the steroid strengths for the FDA-approved topical steroid compositions.
Class 1—superpotent, comprising 7 steroids: clobetasol propionate (0.05%), flurandrenolide (0.05%), betamethasone dipropionate (0.05%), diflorasone diacetate (0.05%), desoxymethasone, and fluocinonide (0.1%).
Class 2—potent, comprising 6 steroids: betamethasone dipropionate (0.05%), mometasone furoate (0.1%), diflorasone diacetate (0.05%), halcinonide (0.1%), fluocinonide (0.05%), desoxymethasone (0.05%-0.25%).
Class 3—upper mid-strength, comprising 3 steroids: fluticasone propionate (0.005%), fluocinonide (0.05%) and betamethasone valerate (0.12%).
Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).
Class 5—lower mid-strength, comprising 7 steroids: fluocinolone acetonide (0.01%), flurandrenolide (0.05%), fluticasone propionate (0.05%), prednicarbate (0.1%), desonide (0.05%), hydrocortisone (0.1%), hydrocortisone valerate (0.2%).
Class 6—mild, comprising only 3 steroids: alclomethasone dipropionate (0.05%), fluocinolone acetonide (0.01%), desonide (0.05%),
Class 7—least potent, comprising only one steroid: hydrocortisone (0.5%, 1%, 2%, 2.5%).

The above corticosteroids are marketed as lotions, creams, solutions, ointments, foam, sprays, gels. Many of the above topical drugs are sold as creams.

### Calcipotriene

Calcipotriene (also known as calcipotriol) is used inter alia. in the topical treatment of several topical disorders, including psoriasis.

Topical calcipotriene and calcipotriene hydrate drugs are FDA-approved as 0.005% solution, cream and aerosol foam dosage forms.

Combinations of 0.005% calcipotriene with 0.064% betamethasone dipropionate are FDA-approved as ointment, suspension and aerosol foam.

### JAK Inhibitors

JAK (Janus kinase) inhibitors (JAKi), also known as jakinibs, are drugs inhibiting the activity of one or more of the Janus kinase family of enzymes (JAK1, JAK2, JAK 1/2, JAK3, TYK2). JAK3 inhibitors have been investigated for the treatment of autoimmune diseases.

Selective JAK3 inhibitors are being developed (Forster M, et al. (September 2017). “Recent advances in JAK3 inhibition: Isoform selectivity by covalent cysteine targeting”. Bioorganic & Medicinal Chemistry Letters. 27 (18): 4229-4237).

A JAK1 inhibitor (PF-04965842, Pfizer) is investigated for treatment of atopic dermatitis and moderate to severe psoriasis (ClinicalTrials.gov).

In some embodiments, baricitinib is a JAK 1/2 inhibitor.

In some embodiment, the at least one JAK inhibitor in the compositions of this invention is selected from a JAK1 inhibitor, a JAK2 inhibitor, a JAK 1/2 inhibitor, a JAK3 inhibitor, a TYK2 inhibitor and combinations thereof.

In some embodiments, there is provided any one of the compositions of this invention, wherein said at least one JAK inhibitor is a pan-JAK inhibitor.

In some embodiments, there is provided any one of the methods of treatment of this invention, wherein said at least one JAK inhibitor is a pan-JAK inhibitor.

In some embodiments the at least one JAK inhibitor is selected from tofacitinib, abrocitinib, ruxolitinib, delgocitinib, oclacitinib, baricitinib, peficitinib and combinations thereof.

The tapinarof/JAKi or tapinarof/JAKi/corticosteroid synergistic or additive effect of the compositions of this invention allows using lower doses of JAK inhibitors and the topical route avoids systemic side-effects on the immune system due to absorption of potentially toxic JAK inhibitors.

### Topical Tapinarof Combination Compositions

Provided herein are compositions, combinations, kits and articles of manufacture that include tapinarof in combination with at least one additional active agent, for treating a skin disorder selected from psoriasis and atopic dermatitis. The compositions, combinations and articles of manufacture can be administered using a variety of routes such as topical application or transdermal application. As one example, provided herein is an article of manufacture that includes tapinarof, at least one additional active agent and a carrier for topical administration, for treating a skin disorder selected from psoriasis and atopic dermatitis.

Also provided herein is an article of manufacture that includes tapinarof and at least one additional active agent, adjusted to a dosage suitable for the treatment of a skin disorder, and a carrier for topical administration. The articles of manufacture provided herein can further contain a label indicating that the composition is for treating a skin disorder as provided herein. The at least one additional active agent may be at least one corticosteroid of a strength suitable for the medical indication, vitamin D analogues, at least one JAK inhibitor or combinations thereof.

In another embodiment the Vitamin D analogue is calcipotriene, paricalcitol or hydrate thereof. In another embodiment, the Vitamin D analogue is calcipotriene or hydrate thereof. In another embodiment, the Vitamin D analogue is paricalcitol or hydrate thereof.

The at least one corticosteroid in the compositions for the treatment of psoriasis may be superpotent (Class 1) or potent (Class 2). Alternatively, the steroid may be of lower potency (Class 3-7), thus minimizing the steroid side-effects, including the risk of pituitary suppression.

The at least one corticosteroid in the compositions for the treatment of atopic dermatitis may be of medium or low potency (Class 4-7).

The articles of manufacture provided herein can further include a delivery system.

The delivery system can be selected from among a variety of vehicles for administering therapeutic agents, as known to those of skill in the art. For example, the delivery system can be selected from among a transdermal patch, a lotion, a cream, an ointment, a gel, a spray, a foam delivery system or an applicator syringe.

Also provided herein are compositions containing tapinarof and at least one additional active agent selected from at least one corticosteroid, vitamin D analogues (such as calcipotriene), at least one JAK inhibitor and combinations thereof and a carrier suitable for topical administration.

The at least one corticosteroid can be selected from among any of those provided herein, incorporated by reference herein, identified by assays as provided herein, or known to those of skill in the art.

Therapeutically effective concentrations for treatment, prevention or amelioration of the symptoms manifested by the skin disorder of tapinarof and at least one additional active agent is mixed with a suitable pharmaceutical carrier or vehicle for topical, transdermal or other routes.

Tapinarof and the at least one additional active agent in the combination compositions are included in an amount effective for reducing the skin disorder for which treatment is contemplated. The concentration of the active agents in the composition will depend on absorption, inactivation, excretion rates of the active compound, the synergistic or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof and/or the at least one additional active agent in the marketed drug currently administered for the treatment of the contemplated skin disorder. The dosage and regimen of administration may be determined by dose finding studies, as known in the art.

Exemplary dosages, strengths and concentrations of tapinarof in the tapinarof combination compositions administered topically, can be in the range of from about or at 0.1%, 0.25%, 0.5%, 1%, 2% or 3% w/w. Typical strengths in the topical combination compositions of this invention are 0.25%, 0.5% or 1% w/w tapinarof. The frequency of administration can be determined empirically. Exemplary frequencies are once daily, twice daily, weekly, bi-weekly or monthly. Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

The resulting compositions may be a solution, suspension, emulsion or the like and are formulated as creams, gels, ointments, emulsions, solutions, elixirs, lotions, suspensions, tinctures, pastes, foams, aerosols, sprays, patches, foams or any other formulation suitable for topical administration.

Pharmaceutical carriers or vehicles suitable for administration of the compounds provided herein include any such carriers known to those skilled in the art to be suitable for the particular mode of administration. In addition, the compounds may be formulated as the sole pharmaceutically active ingredient in the composition or may be combined with other active ingredients. The active agents are included in the carrier in an amount sufficient to exert a therapeutically useful effect i.e., amelioration of the symptoms of the skin disorder, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include those suited for use include lotions, creams, solutions, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

### Tapinarof Combination Compositions for the Treatment of Psoriasis

The at least one corticosteroid in the compositions of this invention for the treatment, prevention or amelioration of psoriasis may be superpotent (Class 1) or potent (Class 2). Alternatively, the steroid may be of lower potency, selected from upper mid-strength (Class 3), mid-strength (Class 4) lower mid-strength (Class 5), mild (Class 6) or least potent (Class 7), thus minimizing the steroid side-effects, including the risk of pituitary suppression.

The superpotent (Class 1) or potent (Class 2) corticosteroids are selected from the following steroids (The percentages in parentheses are the steroid strengths for the FDA-approved topical steroid compositions):
Class 1—superpotent, comprising 7 steroids: clobetasol propionate (0.05%), flurandrenolide (0.05%), betamethasone dipropionate (0.05%), diflorasone diacetate (0.05%), desoxymethasone, and fluocinonide (0.1%).
Class 2—potent, comprising 6 steroids: betamethasone dipropionate (0.05%), mometasone furoate (0.1%), diflorasone diacetate (0.05%), halcinonide (0.1%), fluocinonide (0.05%), desoxymethasone (0.05%-0.25%).

The lower potency steroids are selected from:
Class 3—upper mid-strength, comprising 3 steroids: fluticasone propionate (0.005%), fluocinonide (0.05%) and betamethasone valerate (0.12%).
Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).
Class 5—lower mid-strength, comprising 7 steroids: fluocinolone acetonide (0.01%), flurandrenolide (0.05%), fluticasone propionate (0.05%), prednicarbate (0.1%), desonide (0.05%), hydrocortisone (0.1%), hydrocortisone valerate (0.2%).
Class 6—mild, comprising only 3 steroids: alclomethasone dipropionate (0.05%), fluocinol one acetonide (0.01%), desonide (0.05%).
Class 7—least potent, comprising only one steroid: hydrocortisone (0.5%, 1%, 2%, 2.5%).

In some embodiments, there is provided a combination composition for the treatment, prevention or amelioration of psoriasis, (including plaque psoriasis, or flexural/inverse psoriasis) by topical administration to a subject in need thereof of a composition comprising a therapeutically effective dose of tapinarof and a therapeutically effective dose of at least one additional active agent selected from at least one superpotent (Class 1) or potent (Class 2) corticosteroid, calcipotriene and combinations thereof and a carrier suitable for topical administration.

According to some embodiments, the above at least one superpotent (Class 1) corticosteroid is selected from (the percentages in parentheses are the steroid strengths for the FDA-approved topical steroid compositions) clobetasol propionate (0.05%), flurandrenolide (0.05%), betamethasone dipropionate (0.05%), diflorasone diacetate (0.05%), desoxymethasone, and fluocinonide (0.1%) and combinations thereof and the potent (Class 2) corticosteroid is selected from betamethasone dipropionate (0.05%), mometasone furoate (0.1%), diflorasone diacetate (0.05%), halcinonide (0.1%), fluocinonide (0.05%), desoxymethasone (0.05%-0.25%) and combinations thereof.

In some other embodiments, there is provided a combination composition for the treatment, prevention or amelioration of psoriasis, (including plaque psoriasis, or flexural/inverse psoriasis) by topical administration to a subject in need thereof of a composition comprising a therapeutically effective dose of tapinarof and a therapeutically effective dose of at least one additional active agent selected from at least one corticosteroid of mid-strength (Class 4), lower mid-strength (Class 5), mild (Class 6) or least potent (Class 7), calcipotriene, combinations thereof and a carrier suitable for topical administration.

According to some embodiments, there is provided a combination composition for the treatment, prevention or amelioration of psoriasis, (including plaque psoriasis, or flexural/inverse psoriasis) by topical administration to a subject in need thereof of a composition comprising a therapeutically effective dose of tapinarof, a therapeutically effective dose of at least one corticosteroid of mid-strength (Class 4), lower mid-strength (Class 5), mild (Class 6) or least potent (Class 7), a therapeutically effective dose of calcipotriene, combinations thereof and a carrier suitable for topical administration.

According to some other embodiments, there is provided a combination composition for the treatment, prevention or amelioration of psoriasis, (including plaque psoriasis or flexural/inverse psoriasis) by topical administration to a subject in need thereof of a composition comprising a therapeutically effective dose of tapinarof and a therapeutically effective dose of at least one corticosteroid of mid-strength (Class 4), lower mid-strength (Class 5), mild (Class 6) or least potent (Class 7), combinations thereof and a carrier suitable for topical administration.

In some other embodiments, the strength of the at least one corticosteroid in the compositions for the treatment, prevention or amelioration of psoriasis is 25% w/w, 50% w/w or 75% w/w lower that the strength of the FDA-approved corticosteroids (see above FDA-approved strengths in parentheses).

In some embodiments, there is provided a composition for the treatment, prevention or amelioration of psoriasis, comprising 0.25-2% w/w tapinarof, 0.01-0.05% w/w betamethasone dipropionate and a carrier suitable for topical administration.

In some embodiments, there is provided a composition for the treatment, prevention or amelioration of psoriasis, comprising 0.5% w/w tapinarof, 0.025% w/w betamethasone dipropionate and a carrier suitable for topical administration.

In some embodiments, there is provided a composition for the treatment, prevention or amelioration of psoriasis, comprising 0.25-2% w/w tapinarof, 0.001-0.005% w/w calcipotriene and a carrier suitable for topical administration.

In some embodiments, there is provided a composition for the treatment, prevention or amelioration of psoriasis, comprising 0.5% w/w tapinarof, 0.0025% w/w calcipotriene and a carrier suitable for topical administration.

In some embodiments, there is provided a composition for the treatment, prevention or amelioration of psoriasis, comprising 0.25-2% w/w tapinarof, 0.001-0.005% w/w calcipotriene, 0.01-0.05% betamethasone dipropionate and a carrier suitable for topical administration.

In some embodiments, there is provided a composition for the treatment, prevention or amelioration of psoriasis, comprising 0.5% w/w tapinarof, 0.0025% w/w calcipotriene, 0.025% betamethasone dipropionate and a carrier suitable for topical administration.

The above composition may be a solution, suspension, emulsion or the like and is formulated as a cream, a gel, an ointment, an emulsion, a solution, an elixir, a lotion, a suspension, a tincture, a paste, a foam, an aerosol, a spray, a patch or any other formulation suitable for topical administration.

In some embodiments the composition described herein for the treatment, prevention or amelioration of psoriasis include the main types of psoriasis: plaque, guttate, inverse, pustular, flexural/inverse and erythrodermic. Plaque psoriasis (psoriasis vulgaris) is the most common form of psoriasis.

### Tapinarof Combination Compositions for the Treatment of Atopic Dermatitis

According to some embodiments, there is provided a composition for the treatment, prevention or amelioration of atopic dermatitis, comprising a therapeutically effective dose of tapinarof in combination with a therapeutically effective dose of at least one additional active agent selected from of at least one medium or low potency corticosteroid (Class 4-7), at least one JAK inhibitor, combinations thereof and a carrier suitable for topical administration.

In some embodiments, there is provided a composition for the treatment, prevention or amelioration of atopic dermatitis, comprising a therapeutically effective dose of tapinarof, a therapeutically effective dose of at least one medium or low potency corticosteroid (Class 4-7) and a carrier suitable for topical administration.

In some other embodiments, there is provided a composition for the treatment, prevention or amelioration of atopic dermatitis, comprising a therapeutically effective dose of tapinarof in combination with a therapeutically effective dose of a therapeutically effective dose of a JAK inhibitor.

In some other embodiments, there is provided a composition for the treatment, prevention or amelioration of atopic dermatitis, comprising a therapeutically effective dose of tapinarof, a therapeutically effective dose of at least one medium or low potency corticosteroid (Class 4-7), a therapeutically effective dose of a JAK1 or a JAK2 inhibitor and a carrier suitable for topical administration.

In some embodiments, there is provided a composition for the treatment, prevention or amelioration of atopic dermatitis, comprising 0.25-2% w/w tapinarof, 0.05-0.2% w/w triamcinolone and a carrier suitable for topical administration.

In some other embodiments, there is provided a composition for the treatment, prevention or amelioration of atopic dermatitis, comprising 0.5% w/w tapinarof, 0.1% w/w triamcinolone and a carrier suitable for topical administration.

According to some embodiments, there is provided a composition for the treatment, prevention or amelioration of atopic dermatitis, comprising 0.25-2% w/w tapinarof, 1-2% w/w ruxolitinib phosphate (calculated as base) and a carrier suitable for topical administration.

In some embodiments, there is provided a composition for the treatment, prevention or amelioration of atopic dermatitis, comprising 0.5% w/w tapinarof, 1.5% w/w ruxolitinib phosphate (calculated as base) and a carrier suitable for topical administration.

In some other embodiments, there is provided a composition for the treatment, prevention or amelioration of atopic dermatitis, comprising 0.25-2% w/w tapinarof, 0.05-0.2% w/w triamcinolone, 1-2% w/w ruxolitinib phosphate (calculated as base) and a carrier suitable for topical administration.

Other exemplary compositions of this invention are detailed in Examples 1-6.

According to some embodiments, there is provided a composition for the treatment, prevention or amelioration of atopic dermatitis, comprising 0.5% w/w tapinarof, 0.1% w/w triamcinolone, 1.5% w/w ruxolitinib and a carrier suitable for topical administration.

According to some other embodiments, there is provided a composition for the treatment, prevention or amelioration of atopic dermatitis, comprising 0.5% w/w tapinarof, 0.05% w/w triamcinolone, 0.75% w/w ruxolitinib and a carrier suitable for topical administration.

The above composition may be a solution, suspension, emulsion or the like and is formulated as a cream, a gel, an ointment, an emulsion, a solution, an elixir, a lotion, a suspension, a tincture, a paste, a foam, an aerosol, a spray, a patch or any other formulation suitable for topical administration.

The above Class 4-7 corticosteroids are selected from the following steroids (FDA-approved strengths are indicated in parentheses).
Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).
Class 5—lower mid-strength, comprising 7 steroids: fluocinolone acetonide (0.01%), flurandrenolide (0.05%), fluticasone propionate (0.05%), prednicarbate (0.1%), desonide (0.05%), hydrocortisone (0.1%), hydrocortisone valerate (0.2%).
Class 6—mild, comprising only 3 steroids: alclomethasone dipropionate (0.05%), fluocinolone acetonide (0.01%), desonide (0.05%),
Class 7—least potent, comprising only one steroid: hydrocortisone (0.5%, 1%, 2%, 2.5%).

In some other embodiments, the strength of the at least one corticosteroid in the compositions for the treatment, prevention or amelioration of atopic dermatitis is 25% w/w, 50% w/w or 75% w/w lower that the strength of the FDA-approved corticosteroids (see above FDA-approved strengths in parentheses).

### Methods of Treatment

According to an aspect of the invention, there is provided a method of treatment, prevention or amelioration of a skin disorder which is treatable, preventable and/or alleviated by treatment of a subject in need thereof with a combination composition of tapinarof and at least one additional active agent selected from at least one corticosteroid, vitamin D analogue (calcipotriene), at least one JAK inhibitor and combinations thereof, the method comprising co-administering to a subject in need thereof therapeutically effective amounts of tapinarof and at least one additional active agent, thereby treating, curing or alleviating the skin disorder. The skin disorder is selected from psoriasis and atopic dermatitis.

In some embodiments, the effective amount is a therapeutically effective amount of tapinarof and at least one additional active agent selected from at least one corticosteroid, calcipotriene, at least one JAK inhibitor and combinations thereof, namely an amount which will cure, treat, mitigate or prevent a skin disorder selected from psoriasis and atopic dermatitis.

In some embodiments, co-administration of tapinarof and at least one additional active agent selected from at least one corticosteroid, calcipotriene, at least one JAK inhibitor and combinations thereof exhibit an additive or synergistic effect while treating or alleviating a skin disorder.

In some other embodiments, the co-administration may be made either by administration of a single combination composition, or alternatively by separate administration of a first composition comprising tapinarof and a second composition comprising the at least one additional active agent selected from at least one corticosteroid, calcipotriene, at least one JAK inhibitor and combinations thereof.

In some embodiment tapinarof and the at least one additional active agent are co-administered as two separate compositions. In some embodiment tapinarof and the at least one additional active agent are co-administered as three separate compositions. In some embodiment tapinarof and the at least one additional active agent are co-administered, wherein each active agent is administered as a separate composition. In some embodiment tapinarof and the at least one additional active agent are administered as a single composition combining tapinarof and the at least one additional active agent.

### Tapinarof Composition

In Some Embodiments Provided Herein a Topical Composition Comprising Tapinarof for Co-Administration with Another Composition Comprising an Additional Active Agent.

In some embodiments, provided herein a topical cream composition, comprising from about 0.25% w/w to about 10.0% w/w tapinarof or a pharmaceutically acceptable salt thereof and less than 1% w/w surfactant. In another embodiment, the surfactant is non-ionic. In another embodiment, the surfactant is ionic. In another embodiment, the surfactant is anionic. In another embodiment, the surfactant is cationic. In another embodiment, the surfactant comprises Carbomer Copolymer Type B, polysorbate 80, sorbitan monooleate, polysorbate 20, PEG 100 stearate, polysorbate 60, sodium dodecylsulfate, sodium lauryl sulfate, cetearyl alcohol, dioctyl sodium sulfosuccinate (=docusate sodium), glyceryl oleate, glyceryl stearate, poloxamers or combination thereof.

In some embodiments provided herein a topical cream composition, comprising from about 1% w/w to about 2% w/w tapinarof or a pharmaceutically acceptable salt thereof and less than 1% w/w surfactant. In another embodiment, the surfactant is non-ionic. In another embodiment, the surfactant is ionic. In another embodiment, the surfactant is anionic. In another embodiment, the surfactant is cationic. In another embodiment, the surfactant comprises Carbomer Copolymer Type B, polysorbate 80, sorbitan monooleate, polysorbate 20, PEG 100 stearate, polysorbate 60, sodium dodecylsulfate, sodium lauryl sulfate, cetearyl alcohol, dioctyl sodium sulfosuccinate (=docusate sodium), glyceryl oleate, glyceryl stearate, poloxamers or combination thereof.

In some embodiments, this invention provides a topical cream composition, comprising from about 0.25% w/w to about 10.0% w/w tapinarof or a pharmaceutically acceptable salt thereof, from about 1% w/w to about 20% surfactant, and optionally from about 0.01% to about 1% w/w an anti-oxidant, wherein the surfactant is ionic. In another embodiment, the surfactant is anionic. In another embodiment, the surfactant is cationic.

Other known tapinarof formulations (e.g. see U.S. Pat. No. 10,195,160) contain non-ionic surfactants at concentration of 1%-20%. For example in U.S. Pat. No. 10,195,160 the formulations contain more than 7% (˜11.6%, see e.g Table 8 formulation 21) of a non-ionic surfactant. Surfactants are known to be irritant and create sensitivity. It is advisable to use low concentration that is still effective not irritant and will stabilize the formulation. While low concentrations may cause stability problems, it is still advisable to seek formulations with low concentrations of surfactants and provide safe, stable and effective formulation. Surprisingly, the present invention provides a composition comprising tapinarof with lower concentrations of less than 1% of surfactant that provides stable formulations that are effective and safe and do not cause any irritations. These formulations contain non-ionic surfactants of less than 1% or non-irritant ionic surfactants, at concentrations of less than 1% or higher.

In another aspect, the present invention also provides tapinarof compositions containing non-irritant ionic surfactants, at concentrations of less than 1% or higher. In another embodiment, the ionic surfactant is anionic. In another embodiment, the ionic surfactant is cationic.

In some embodiments, an anionic surfactant is selected from the group consisting of alkyl sulfates (e.g., sodium lauryl sulfate, ammonium lauryl sulfate and ammonium laureth sulfate); sulfosuccinates (e.g., disodium lauryl sulfosuccinate, disodium laureth sulfosuccinate, sodium dioctyl sulfosuccinate and their mixtures with sulfonic acids and lauramidopropyl betaine; alkyl benzene sulfonates (e.g., sodium tosylate, cumene sulfonate, toluene sulfonic acid, xylene sulfonic acid, cumene sulfonic acid and salts (e.g., sodium, potassium, calcium, ammonium) thereof); acyl methyl taurates (e.g., sodium methyl lauroyl taurate and sodium methyl cocoyl taurate); acyl sarcocinates (e.g., sodium lauroyl sarcosinate, sodium cocoyl sarcosinate and sodium myristoyl sarcosinate); isethionates (e.g., sodium butyl isethionate, sodium capryloyl isethionate and sodium lauroyl isethionate); propyl peptide condensates; monoglyceride sulfates; ether sulfonates, fatty acid salts (e.g., sodium stearoyl lactylate), dioctyl sulfosuccinate, dioctyl sodium sulfosuccinate (=docusate sodium), Carbomer Copolymer Type B (Pemulen®TR-1) and polyacrylic acid polymers.

In another embodiment, the anionic surfactant comprises Carbomer Copolymer Type B (Pemulen®TR-1), sodium lauryl sulfate, sodium dodecylsulfate, or dioctyl sodium sulfosuccinate (docusate sodium). In another embodiment, the anionic surfactant is Carbomer Copolymer Type B (Pemulen®TR-1). In another embodiment, the anionic surfactant is sodium lauryl sulfate. In another embodiment, the anionic surfactant is docusate sodium.

In some embodiments, a cationic surfactant is selected from the group consisting of quaternary ammonium compounds (e.g., benzalkonium chloride, stearalkonium chloride, centrimonium chloride, and trimethyl ammonium methyl sulfates).

In another embodiment, non-ionic surfactant comprises polysorbate 80, sorbitan monooleate, polysorbate 20, polysorbate 60, cetearyl alcohol, glyceryl oleate, glyceryl stearate, ethoxylated fatty alcohol, ethers, PEG castor oils, PEG esters, PEG 100 stearate, propylene glycol esters (e.g. propylene glycol laurate, propylene glycol palmitostearate, propylene glycol ricinoleate and propylene glycol stearate), glyceryl esters and derivatives (e.g. glyceryl behenate, glyceryl dibehenate, glyceryl dioleate, glyceryl distearate, glyceryl isostearate, glyceryl laurate, glyceryl linoleate, glyceryl monostearate, glyceryl oleate, glyceryl palmitate, glyceryl ricinoleate, glyceryl stearate, PEG-23 glyceryl cocoate, PEG-6 caprylic/capric glycerides, PEG-7 glyceryl cocoate, polyglyceryl-10 diisostearate, polyglyceryl-2 diisostearate, polyglyceryl-3 diisostearate and polyglyceryl-6 diisostearate, PEG-12 glyceryl laurate, PEG-120 glyceryl stearate), polymeric ethers (e.g. poloxamer 124, poloxamer 181, poloxamer 182, poloxamer 184, poloxamer 188, poloxamer 237, poloxamer 331, poloxamer 338 and poloxamer 407). Sorbitan derivatives (e.g. polysorbate 20, polysorbate 40, polysorbate 60, polysorbate 65, polysorbate 80, sorbitan isostearate, sorbitan monolaurate, sorbitan monooleate, sorbitan monopalmitate, sorbitan monostearate, sorbitan sesquioleate, sorbitan trioleate and sorbitan tristearate), fatty alcohols (e.g. isostearyl alcohol, caprylyl alcohol, decyl alcohol, lauryl alcohol, myristyl alcohol, behenyl alcohol, lanolin alcohol, arachidyl alcohol, oleyl alcohol, palm alcohol, isocetyl alcohol, cetyl alcohol, stearyl alcohol and cetearyl alcohol), emulsifying waxes (e.g. mixture of cetearyl alcohol and polysorbate 60 or “Polawax NF™, and poloxamers or combination thereof.

In some embodiments, the composition provided herein further comprises from about 0.1% to about 4% w/w gelling agent. In some embodiments, the composition provided herein comprises from about 0.01% to about 1% w/w anti-oxidant.

In some embodiments, the composition provided herein comprises from about 0.1% w/w to about 20% w/w surfactant. In some embodiments, the composition provided herein comprises less than 5% w/w of the surfactant. In some embodiments, the composition provided herein comprises less than 1% w/w of the surfactant. In other embodiments, the surfactant is in an amount of about 5% w/w to about 20% w/w. In other embodiments, the surfactant is in an amount of about 10% w/w to about 20% w/w. In other embodiments, the surfactant is in an amount of about 15% w/w to about 20% w/w. In other embodiments, the surfactant is in an amount of about 5% w/w to about 10% w/w. In other embodiments, the surfactant is in an amount of about 5% w/w. In other embodiments, the surfactant is in an amount of about 1% w/w to about 5% w/w. In other embodiments, the composition comprises the surfactant in an amount of about 0.1% to about 5% w/w. In other embodiments, the surfactant is in an amount of about 0.1% to about 4% w/w. In other embodiments, the surfactant is in an amount of about 0.1% to about 3% w/w. In other embodiments, the surfactant is in an amount of about 0.1% to about 2% w/w. In other embodiments, the surfactant is in an amount of about 0.1% to about 1.5% w/w. In other embodiments, the surfactant is in an amount of about 0.1 to about 1% w/w. In other embodiments, the surfactant is in an amount of about 0.1 to about 0.5% w/w. In other embodiments, the surfactant is in an amount of about 0.1 to about 0.3% w/w. In other embodiments, the surfactant is in an amount of about 0.1 to about 0.2% w/w. In some other embodiments, the surfactant is in an amount of about 0.4% w/w. In other embodiments, the surfactant is in an amount of about 0.2% to about 0.8% w/w. In other embodiments, the surfactant is ionic. In other embodiments, the surfactant is anionic. In other embodiments, the surfactant is cationic. In other embodiments, the surfactant is non-ionic. In other embodiments, the surfactant is Carbomer Copolymer Type B. In other embodiments, the surfactant is in an amount of about 0.4% w/w Carbomer Copolymer Type B. In other embodiments, the surfactant is in an amount of about 0.2% to about 0.8% w/w Carbomer Copolymer Type B. In other embodiments, the surfactant is docusate sodium. In other embodiments, the surfactant is sodium lauryl sulfate.

In some embodiments, the composition provided herein comprises a gelling agent. In some embodiments, the composition further comprises an additional gelling agent. In some embodiments, the gelling agent and the additional gelling agent are each independently selected from a group consisting of a carbomer, Natrosol hydroxyethylcellulose, starch, pectin, gelatin, agar, alginic acid and salts thereof, carboxymethylcellulose, hydroxypropylmethylcellulose and any combination thereof. In other embodiments, the gelling agent or the additional gelling agent is Carbomer Homopolymer Type C (Carbopol®980). In other embodiments, the gelling agent or the additional gelling agent is Carbomer Homopolymer Type C (Carbopol®980), in an amount of about 0.1% to about 4% w/w. In other embodiments, the gelling agent or the additional gelling agent is Carbomer Homopolymer Type C (Carbopol®980), in an amount of 1% w/w, 2% w/w, 3% w/w or 4% w/w.

In some embodiments, the gelling agent and/or additional gelling agent is in an amount of about 0.1% to about 4% w/w. In other embodiments, the gelling agent and/or additional gelling agent is in an amount of about 0.1% to about 2% w/w. In other embodiments, the gelling agent and/or additional gelling agent is in an amount of about 0.1% to about 1% w/w. In other embodiments, the gelling agent and/or additional gelling agent is in an amount of about 0.1% to about 0.5% w/w. In other embodiments, the gelling agent and/or additional gelling agent is in an amount of about 0.5% to about 4% w/w. In other embodiments, the gelling agent and/or additional gelling agent is in an amount of about 0.5% to about 2% w/w. In other embodiments, the gelling agent and/or additional gelling agent is in an amount of about 0.5% to about 1% w/w. In other embodiments, the gelling agent and/or additional gelling agent is in an amount of about 1% to about 4% w/w. In other embodiments, the gelling agent and/or additional gelling agent is in an amount of about 1% to about 2% w/w.

In some embodiments, the composition further comprises an additional surfactant. In some embodiments, the additional surfactant comprises Carbomer Copolymer Type B, polysorbate 80, sorbitan monooleate, polysorbate 20, PEG 100 stearate, polysorbate 60, sodium dodecylsulfate, sodium lauryl sulfate, cetearyl alcohol, dioctyl sodium sulfosuccinate (=docusate sodium), glyceryl oleate, glyceryl stearate or poloxamers or any combination thereof.

In some embodiments, the additional surfactant is in an amount of about 0.1% to about 4% w/w. In other embodiments, the additional surfactant is in an amount of about 0.1% to about 2% w/w. In other embodiments, the additional surfactant is in an amount of about 0.1% to about 1.5% w/w. In other embodiments, the additional surfactant is in an amount of about 0.1% to about 1% w/w. In other embodiments, the additional surfactant is in an amount of about 0.1% to about 0.5% w/w. In other embodiments, the additional surfactant is in an amount of about 0.5% to about 4% w/w. In other embodiments, the additional surfactant is in an amount of about 0.5% to about 2% w/w. In another embodiment, the additional surfactant is in an amount of about 0.5% to about 1.5% w/w. In another embodiment, the additional surfactant is in an amount of about 0.5% to about 1% w/w. In another embodiment, the additional surfactant is in an amount of about 1% to about 4% w/w. In another embodiment, the additional surfactant is in an amount of about 1% to about 2% w/w. Each possibility represents a separate embodiment of this invention.

In some embodiments, the composition further comprises from about 0.1% to about 1.5% w/w preservative. In some embodiments, the preservative is selected from benzoic acid, methylparaben, phenoxyethanol, imidurea, chlorocresol, propyl paraben, benzalkonium chloride, benzyl alcohol, imidazolidinyl urea, sodium benzoate, sorbic acid, thimerosal sorbic acid, sodium sorbate, sodium sulfite, ethanol, tocopherols and any combination thereof. In other embodiments, the preservative is methylparaben. In other embodiments, the preservative is phenoxyethanol. In other embodiments, the preservative is a combination of methylparaben and phenoxyethanol. In other embodiments, the preservative is methylparaben in an amount of 0.3% w/w. In other embodiments, the preservative is phenoxyethanol in an amount of 0.5% w/w. In other embodiments, the preservative is a combination of methylparaben in an amount of 0.3% w/w and phenoxyethanol in an amount of 0.5% w/w.

In some embodiments, the composition further comprises from about 0.1% to about 1.5% w/w preservative. In other embodiments, the preservative is in an amount of about 0.1% to about 1% w/w. In other embodiments, the preservative is in an amount of about 0.1% to about 0.5% w/w. In other embodiments, the preservative is in an amount of about 0.5% to about 1.5% w/w. In other embodiments, the preservative is in an amount of about 0.5% to about 1% w/w. In other embodiments, the preservative is in an amount of 0.5%. In other embodiments, the preservative is in an amount of 0.3%.

In some embodiments, the composition further comprises penetration enhancer. In some embodiments, the penetration enhancer is selected from dimethylsulfoxide (DMSO), diethylene glycol monoethyl ether (Transcutol), methylsulfonylmethane (MSM), propylene glycol, dimethyl isosorbide, oleic acid, oleyl alcohol, ethanol, isopropyl alcohol, a polyethylene glycol, hexylene glycol, glycofurol, isopropyl myristate, glycerin, mono- and di-carboxylic acid esters and any combination thereof. In other embodiments, the penetration enhancer is diethylene glycol monoethyl ether (Transcutol). In other embodiments, the penetration enhancer is propylene glycol. In other embodiments, the penetration enhancer is diethylene glycol monoethyl ether (Transcutol), in an amount of 5% w/w. In other embodiments, the penetration enhancer is propylene glycol in an amount of 15% w/w. In other embodiments, the penetration enhancer is a combination of diethylene glycol monoethyl ether (Transcutol), in an amount of 5% w/w and propylene glycol in an amount of 15% w/w. In some embodiments, in addition and/or instead said penetration enhancer—water is added to the composition. In another embodiment, the penetration enhancer is not dimethylsulfoxide (DMSO).

In some embodiments, the composition comprises a penetration enhancer in an amount of about 0.1% to about 20% w/w. In other embodiments, the penetration enhancer is in an amount of about 0.1% to about 15% w/w. In other embodiments, the penetration enhancer is in an amount of about 0.1% to about 10% w/w. In other embodiments, the penetration enhancer is in an amount of about 0.1% to about 5% w/w. In other embodiments, the penetration enhancer is in an amount about of 0.1% to about 2% w/w. In other embodiments, the penetration enhancer is in an amount of about 0.1% to about 1% w/w. In other embodiments, the penetration enhancer is in an amount of about 0.1% to about 0.5% w/w. In another embodiment other embodiments, the penetration enhancer is in an amount of about 0.5% to about 20% w/w. In other embodiments, the penetration enhancer is in an amount of about 0.5% to about 15% w/w. In other embodiments, the penetration enhancer is in an amount of about 0.5% to about 10% w/w. In other embodiments, the penetration enhancer is in an amount of about 0.5% to about 5% w/w. In other embodiments, the penetration enhancer is in an amount of about 0.5% to about 2% w/w. In other embodiments, the penetration enhancer is in an amount of about 0.5% to about 1% w/w. In other embodiments, the penetration enhancer is in an amount of about 1% to about 20% w/w. In other embodiments, the penetration enhancer is in an amount of about 1% to about 15% w/w. In other embodiments, the penetration enhancer is in an amount of about 1% to about 10% w/w. In other embodiments, the penetration enhancer is in an amount of about 1% to about 5% w/w. In other embodiments, the penetration enhancer is in an amount of about 1% to about 2% w/w. In other embodiments, the penetration enhancer is in an amount of about 2% to about 20% w/w. In other embodiments, the penetration enhancer is in an amount of about 2% to about 15% w/w. In other embodiments, the penetration enhancer is in an amount of about 2% to about 10% w/w. In other embodiments, the penetration enhancer is in an amount of about 2% to about 5% w/w. In other embodiments, the penetration enhancer is in an amount of about 5% to about 20% w/w. In other embodiments, the penetration enhancer is in an amount of about 5% to about 15% w/w. In other embodiments, the penetration enhancer is in an amount of about 5% to about 10% w/w. In other embodiments, the penetration enhancer is in an amount of about 10% to about 20% w/w. In other embodiments, the penetration enhancer is in an amount of about 10% to about 15% w/w. In other embodiments, the penetration enhancer is in an amount of about 15% to about 20% w/w. In other embodiments, the penetration enhancer is in an amount of 5% w/w, 6% w/w, 7% w/w, 8% w/w, 9% w/w, 10% w/w, 11% w/w, 12% w/w, 13% w/w, 14% w/w, 15% w/w, 16% w/w, 17% w/w, 18% w/w, 19% w/w, 20% w/w or any ranges thereof.

In some embodiments, the composition further comprises an emollient. In one embodiment, non-limiting examples of the emollient include: coconut fatty acid diethanolamide, petrolatum, mineral oil light, castor oil and any combination thereof.

In some embodiments the emollient is in a weight of about 0.01 to about 2% w/w. In one embodiment, the emollient is in a weight of about 0.01 to about 1.5% w/w. In another embodiment, the emollient is in a weight of about 0.01 to about 1% w/w. In another embodiment, the emollient is in a weight of about 0.01 to about 0.5% w/w. In another embodiment, the emollient is in a weight of about 0.01 to about 0.2% w/w. In another embodiment, the emollient is in a weight of about 0.01 to about 0.1% w/w. In another embodiment, the emollient is in a weight of about 0.1 to about 2% w/w. In another embodiment, the emollient is in a weight of about 0.1 to about 1.5% w/w. In another embodiment, the emollient is in a weight of about 0.1 to about 1% w/w. In another embodiment, the emollient is in a weight of about 0.1 to about 0.5% w/w. In another embodiment, the emollient is in a weight of about 0.1 to about 0.2% w/w. In another embodiment, the emollient is in a weight of about 0.2 to about 2% w/w. In another embodiment, the emollient is in a weight of about 0.2 to about 1.5% w/w. In another embodiment, the emollient is in a weight of about 0.2 to about 1% w/w. In another embodiment, the emollient is in a weight of about 0.2 to about 0.5% w/w. In another embodiment, the emollient is in a weight of about 0.5 to about 2% w/w. In another embodiment, the emollient is in a weight of about 0.5 to about 1.5% w/w. In another embodiment, the emollient is in a weight of about 0.5 to about 1% w/w. In another embodiment, the emollient is in a weight of about 1 to about 2% w/w. In another embodiment, the emollient is in a weight of about 1.5 to about 2% w/w. In another embodiment, the emollient is in a weight of about 1 to about 1.5 w/w.

In some embodiments, the composition further comprises a chelating agent. In one embodiment, non-limiting examples of the chelating agent include: ethylenediaminetetraacetic acid (EDTA), nitrolotriacetic acid (NTA) and citric acid.

In some embodiments the chelating agent is in a weight of about 0.01 to about 2% w/w. In one embodiment, the chelating agent is in a weight of about 0.01 to about 1.5% w/w. In another embodiment, the chelating agent is in a weight of 0.1% w/w.

In some embodiments, the composition further comprises a pH adjusting agent. In one embodiment, non-limiting examples of the pH adjusting agent include: sodium hydroxide, potassium hydroxyide, hydrochloric acid, sodium acetate, acetic acid, ammonium acetate, ammonium sulfate, ammoniu hydroxide, arginine, aspartic acid and salts thereof, bicarbonate salts, carbonate salts, citric acid and salts thereof, diethanolamine, glycine, lysine, boric acid and salts thereof and phosphate salts. In some embodiments the pH adjusting agent is in a weight of about 0.01 to about 5 w/w. In one embodiment the pH adjusting agent is in a weight of about 0.01 to about 2% w/w. In another embodiment, the pH adjusting agent is in a weight of 1% w/w.

In some embodiments, the purity of the tapinarof is greater than 97%. In some embodiments, the purity of the tapinarof is greater than 98%. In some embodiments, the purity of the tapinarof is greater than 99%. In some embodiments, the purity of the tapinarof is greater than 99.5%. In some embodiments, the purity of the tapinarof is between 97% and 99.99%. In some embodiments, the purity of the tapinarof is between 99% and 99.99%.

In some embodiments, the composition comprises tapinarof in an amount of about 0.25% to about 10% w/w. In other embodiments, the tapinarof is in an amount of about 0.25% to about 5% w/w. In other embodiments, the tapinarof is in an amount of about 0.25% to about 2% w/w. In other embodiments, the tapinarof is in an amount of about 0.25% to about 1% w/w. In other embodiments, the tapinarof is in an amount of about 0.25% to about 0.5% w/w. In other embodiments, the tapinarof is in an amount of about 0.5% to about 10% w/w. In other embodiments, the tapinarof is in an amount of about 0.5% to about 5% w/w. In other embodiments, the tapinarof is in an amount of about 0.5% to about 2% w/w. In another embodiment, the tapinarof is in an amount of about 0.5% to about 1% w/w. In other embodiments, the tapinarof is in an amount of about 1% to about 10% w/w. In another embodiment, the tapinarof is in an amount of about 1% to about 5% w/w. In other embodiments, the tapinarof is in an amount of about 1% to about 2% w/w. In other embodiments, the tapinarof is in an amount of about 2% to about 10% w/w. In other embodiments, the tapinarof is in an amount of about 2% to about 5% w/w. In other embodiments, the tapinarof is in an amount of about 5% to about 10% w/w. In other embodiments, the tapinarof is in an amount of 1.5% w/w. In other embodiments, the tapinarof is in an amount of 1% w/w. In other embodiments the tapinarof is in an amount from about 1% w/w to about 2% w/w. In other embodiments, the tapinarof is in an amount of 0.75% w/w, 1% w/w, 2% w/w, 3% w/w, 4% w/w, 5% w/w, 6% w/w, 7% w/w, 8% w/w, 9% w/w, 10% w/w or any ranges thereof.

In some embodiments, the composition comprises from about 0.01% to about 1% w/w an anti-oxidant. In some embodiments, the anti-oxidant is selected from the group consisting from butylated hydroxy toluene (BHT), parabens, propyl gallate, ascorbic acid, flavones, flavanones, flavonols, stilbenoids, gallic acid, cinnamic acid, ellagic acid, salicylic acid, curcumin, eugenol, citric acid, tocopherol, vitamin E, tocopherol polyethylene glycol succinate (TPGS), and any combination thereof. In other embodiments, the anti-oxidant is butylated hydroxy toluene (BHT). In other embodiments, the anti-oxidant is butylated hydroxy toluene (BHT), in an amount of 0.1% w/w.

In some embodiments, the composition comprises from about 0.01% to about 1% w/w an anti-oxidant. In other embodiments, the anti-oxidant is in an amount of about 0.01% to about 0.02% w/w. In other embodiments, the anti-oxidant is in an amount of about 0.01% to about 0.05% w/w. In other embodiments, the anti-oxidant is in an amount of about 0.01% to about 0.1% w/w. In other embodiments, the anti-oxidant is in an amount of about 0.01% to about 0.2% w/w. In other embodiments, the anti-oxidant is in an amount of about 0.01% to about 0.5% w/w. In other embodiments, the anti-oxidant is in an amount of about 0.02% to about 1% w/w. In other embodiments, the anti-oxidant is in an amount of about 0.02% to about 0.05% w/w. In other embodiments, the anti-oxidant is in an amount of about 0.02% to about 0.1% w/w. In other embodiments, the anti-oxidant is in an amount of about 0.02% to about 0.2% w/w. In other embodiments, the anti-oxidant is in an amount of about 0.02% to about 0.5% w/w. In other embodiments, the anti-oxidant is in an amount of about 0.05% to about 1% w/w. In other embodiments, the anti-oxidant is in an amount of about 0.05% to about 0.1% w/w. In other embodiments, the anti-oxidant is in an amount of about 0.05% to about 0.2% w/w. In other embodiments, the anti-oxidant is in an amount of about 0.05% to about 0.5% w/w. In other embodiments, the anti-oxidant is in an amount of about 0.1% to about 1% w/w. In other embodiments, the anti-oxidant is in an amount of about 0.1% to about 0.2% w/w. In other embodiments, the anti-oxidant is in an amount of about 0.1% to about 0.5% w/w. In other embodiments, the anti-oxidant is in an amount of about 0.2% to about 0.5% w/w. In other embodiments, the anti-oxidant is in an amount of about 0.2% to about 1% w/w. In other embodiments, the anti-oxidant is in an amount of about 0.5% to about 1% w/w. In other embodiments, the anti-oxidant is in an amount of 0.5%. In another embodiment, the anti-oxidant is in an amount of 0.1%.

Pharmaceutical carriers or vehicles suitable for preparation of the compositions provided herein include any such carriers known to those skilled in the art to be suitable for the particular mode of administration.

Pharmaceutical carriers or vehicles suitable for administration of the active agents provided herein include any such carriers known to those skilled in the art to be suitable for the particular mode of administration. In addition, the active agents may be formulated as the sole pharmaceutically active agent in the composition or may be combined with other active agents. The active agents are included in the carrier in an amount sufficient to exert a therapeutically useful effect. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles are organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

In one embodiment, the composition according to the invention is a cream. By addition of a fatty or oily phase, in an aqueous phase (O/W) or conversely (W/O), the composition can be in a form of suspensions or emulsions of soft, semi-liquid or solid consistency of a cream, or alternatively multiple emulsions (W/O/W or O/W/O), microemulsions, microcapsules, microparticles or vesicular dispersions of ionic and/or nonionic type, or wax/aqueous phase dispersions. In other embodiment, the composition according to the invention is a cream or an emulsion.

### Medical Treatment Methods Using Tapinarof Composition

In some embodiments, this invention provides a method of treating a skin disorder in a patient in need thereof, said method comprises administering to said patient the topical cream composition as described herein, wherein said skin disorder is selected from psoriasis, toxic and allergic contact eczema, atopic eczema, seborrheic eczema, lichen simplex, sunburn, pruritus (e.g. in the genitoanal region), alopecia areata, hypertrophic scars, discoid lupus erythematosus, follicular and extensive pyodermas, acne, rosacea, atopic dermatitis, prurigo nodularis and hidradenitis suppurativa. In other embodiments, the acne is selected from acne vulgaris, papulopustular acne and nodular acne. In other embodiments, the rosacea is selected from erythematotelangiectatic rosacea, papulopustular rosacea, rhinophyma rosacea and ocular rosacea.

In some embodiments, this invention provides a method of treating a skin disorder in a patient in need thereof, said method comprises administering to said patient the topical cream composition comprising from about 1% w/w to about 2% w/w tapinarof or a pharmaceutically acceptable salt thereof and less than 1% w/w surfactant;
wherein said skin disorder is selected from psoriasis, toxic and allergic contact eczema, atopic eczema, seborrheic eczema, lichen simplex, sunburn, pruritus (e.g. in the genitoanal region), alopecia areata, hypertrophic scars, discoid lupus erythematosus, follicular and extensive pyodermas, acne, rosacea, atopic dermatitis, prurigo nodularis and hidradenitis suppurativa. In another embodiment, the surfactant is ionic or non-ionic. In another embodiment, the surfactant is anionic. In another embodiment, the surfactant is cationic.

In some embodiments, this invention provides a method of treating a skin disorder in a patient in need thereof, said method comprises administering to said patient the topical cream composition comprising from about 0.25% w/w to about 10.0% w/w tapinarof or a pharmaceutically acceptable salt thereof and less than 1% w/w surfactant;
wherein said skin disorder is selected from psoriasis, toxic and allergic contact eczema, atopic eczema, seborrheic eczema, lichen simplex, sunburn, pruritus (e.g. in the genitoanal region), alopecia areata, hypertrophic scars, discoid lupus erythematosus, follicular and extensive pyodermas, acne, rosacea, atopic dermatitis, prurigo nodularis and hidradenitis suppurativa. In another embodiment, the surfactant is ionic or non-ionic. In another embodiment, the surfactant is anionic. In another embodiment, the surfactant is cationic.

In some embodiments, this invention provides a method of treating a skin disorder in a patient in need thereof, said method comprises administering to said patient the topical cream composition comprising from about 0.25% w/w to about 10.0% w/w tapinarof or a pharmaceutically acceptable salt thereof, from about 0.1% w/w to about 20% surfactant, and optionally from about 0.01% to about 1% w/w an anti-oxidant, wherein the surfactant is ionic;
wherein said skin disorder is selected from psoriasis, toxic and allergic contact eczema, atopic eczema, seborrheic eczema, lichen simplex, sunburn, pruritus (e.g. in the genitoanal region), alopecia areata, hypertrophic scars, discoid lupus erythematosus, follicular and extensive pyodermas, acne, rosacea, atopic dermatitis, prurigo nodularis and hidradenitis suppurativa. In another embodiment, the surfactant is anionic. In another embodiment, the surfactant is cationic.

In some embodiments, the methods and compositions provided herein are for use in the treatment of psoriasis. In some embodiments, the methods and compositions provided herein are for use in the treatment of toxic and allergic contact eczema. In some embodiments, the methods and compositions provided herein are for use in the treatment of atopic eczema. In some embodiments, the methods and compositions provided herein are for use in the treatment of seborrheic eczema. In some embodiments, the methods and compositions provided herein are for use in the treatment of lichen simplex. In some embodiments, the methods and compositions provided herein are for use in the treatment of sunburn, pruritus (e.g. in the genitoanal region). In some embodiments, the methods and compositions provided herein are for use in the treatment of alopecia areata. In some embodiments, the methods and compositions provided herein are for use in the treatment of hypertrophic scars. In some embodiments, the methods and compositions provided herein are for use in the treatment of discoid lupus erythematosus. In some embodiments, the methods and compositions provided herein are for use in the treatment of follicular and extensive pyodermas. In some embodiments, the methods and compositions provided herein are for use in the treatment of acne. In some embodiments, the methods and compositions provided herein are for use in the treatment of rosacea. In some embodiments, the methods and compositions provided herein are for use in the treatment of atopic dermatitis. In some embodiments, the methods and compositions provided herein are for use in the treatment of prurigo nodularis. In some embodiments, the methods and compositions provided herein are for use in the treatment of hidradenitis suppurativa.

Dosage frequencies can be gradually attenuated over time and maintained at a steady dose suitable for long-term—six months, 1 year, 5 years, 10 years or more, up to lifelong administration to control the symptoms of the hidradenitis suppurativa. For example, dosage administration can begin at from twice a day, to once a day, to two times a week, to once a week, to once every two weeks or less frequent than once every two weeks.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of said skin disorders. For example, dosage administration can begin at from twice a day, to once a day, to two times a week, to once a week, to once every two weeks or less frequent than once every two weeks.

### Regimen of Administration of the Topical Tapinarof Combination Compositions

Therapeutically effective concentrations of tapinarof and at least one additional active agent in the compositions of this invention, for treatment, prevention or amelioration of the symptoms manifested by the skin disorder are determined by empirical methods known in the art.

Tapinarof and the at least one additional active agent in the combination compositions are included in an amount effective for reducing the skin disorder for which treatment is contemplated. The concentration of the active agents in the composition will depend on absorption, inactivation, excretion rates of the active compound, the synergistic or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof and/or the at least one additional active agent in the marketed drug currently administered for the treatment of the contemplated skin disorder. The dosage and regimen of administration may be determined by dose finding studies, as known in the art.

Exemplary dosages, strengths and concentrations of tapinarof in the tapinarof combination compositions administered topically, can be in the range of between 0.25-5% w/w or between 0.25-2% w/w or at 0.1%, 0.25%, 0.5%, 1%, 2%, 3%, 4% or 5% w/w.

Typical strengths in the topical combination compositions of this invention are 0.25%, 0.5% or 1% w/w tapinarof.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

Treatment of Psoriasis with Tapinarof Combinations

According to one aspect of this invention, psoriasis is treated with tapinarof combinations with at least one corticosteroid and/or calcipotriene.

In another embodiment, psoriasis is treated with between 0.25-5% w/w tapinarof with at least one corticosteroid and/or calcipotriene. In another embodiment, psoriasis is treated with between 0.25-2% w/w tapinarof with at least one corticosteroid and/or calcipotriene.

The corticosteroid used is of very high or high potency (Class 1 or 2, see above). Alternatively, the corticosteroid is selected from a lower group of potency (Class 3-7), which has milder side-effects and lower risk of pituitary suppression.

Due to the synergistic or additive effect with tapinarof, the corticosteroid can also be used at a lower strength (steroid-sparing) than the US-marketed topical drugs used for the treatment of psoriasis.

According to some embodiments, there is provided a method of treatment, prevention or amelioration of psoriasis by topical administration to a subject in need thereof of a composition comprising a therapeutically effective dose of tapinarof and a therapeutically effective dose of at least one additional active agent selected from at least one superpotent (Class 1) or potent (Class 2) corticosteroid, calcipotriene and combinations thereof and a carrier suitable for topical administration.

According to some embodiments, the above at least one superpotent (Class 1) corticosteroid is selected from clobetasol propionate (0.05%), flurandrenolide (0.05%), betamethasone dipropionate (0.05%), diflorasone diacetate (0.05%), desoxymethasone, and fluocinonide (0.1%) and combinations thereof and the potent (Class 2) corticosteroid is selected from betamethasone dipropionate (0.05%), mometasone furoate (0.1%), diflorasone diacetate (0.05%), halcinonide (0.1%), fluocinonide (0.05%), desoxymethasone (0.05%-0.25%) and combinations thereof.

According to some embodiments, there is provided a method of treatment, prevention or amelioration of psoriasis by topical administration to a subject in need thereof of a composition comprising a therapeutically effective dose of tapinarof and a therapeutically effective dose of at least one additional active agent selected from at least one corticosteroid of mid-strength (Class 4), lower mid-strength (Class 5), mild (Class 6) or least potent (Class 7), calcipotriene, combinations thereof and a carrier suitable for topical administration.

According to some embodiments, there is provided a method of treatment, prevention or amelioration of psoriasis by topical administration to a subject in need thereof of a composition comprising a therapeutically effective dose of tapinarof, a therapeutically effective dose of at least one corticosteroid of mid-strength (Class 4), lower mid-strength (Class 5), mild (Class 6) or least potent (Class 7), a therapeutically effective dose of calcipotriene, combinations thereof and a carrier suitable for topical administration.

According to some embodiments, there is provided a method of treatment of psoriasis by topical administration to a subject in need thereof of a composition comprising a therapeutically effective dose of tapinarof and a therapeutically effective dose of at least one corticosteroid of mid-strength (Class 4), lower mid-strength (Class 5), mild (Class 6) or least potent (Class 7), combinations thereof and a carrier suitable for topical administration.

In some other embodiments, the strength of the at least one corticosteroid in the compositions for the treatment of psoriasis is 25% w/w, 50% w/w or 75% w/w lower that the strength of the FDA-approved corticosteroids (see above FDA-approved strengths in parentheses).

In some embodiments, there is provided a composition for the treatment, prevention or amelioration of psoriasis, comprising 1.0% w/w tapinarof, 0.005% w/w calcipotriene, 0.05% betamethasone dipropionate and a carrier suitable for topical administration.

In some embodiments, there is provided a composition for the treatment, prevention or amelioration of psoriasis, comprising 0.5% tapinarof, 0.0025% calcipotriene, 0.025% betamethasone dipropionate and a carrier suitable for topical administration.

The above composition may be a solution, suspension, emulsion or the like and is formulated as a cream, a gel, an ointment, an emulsion, a solution, an elixir, a lotion, a suspension, a tincture, a paste, a foam, an aerosol, a spray, a patch or any other formulation suitable for topical administration.

According to some embodiments, there is provided a method of treatment, prevention or amelioration of psoriasis by topical administration to a subject in need thereof of a composition comprising a therapeutically effective dose of tapinarof in combination with a therapeutically effective dose of at least one additional active agent selected from of at least one superpotent or potent corticosteroid (Class 1-2), at least one JAK inhibitor and combinations thereof.

According to some embodiments, there is provided a method of treatment, prevention or amelioration of psoriasis by topical administration to a subject in need thereof of a composition comprising between 0.25-5% w/w tapinarof in combination with a therapeutically effective dose of at least one additional active agent selected from of at least one superpotent or potent corticosteroid (Class 1-2), at least one JAK inhibitor and combinations thereof.

According to some embodiments, there is provided a method of treatment, prevention or amelioration of psoriasis by topical administration to a subject in need thereof of a composition comprising between 0.25-2% w/w tapinarof in combination with a therapeutically effective dose of at least one additional active agent selected from of at least one superpotent or potent corticosteroid (Class 1-2), at least one JAK inhibitor and combinations thereof.

In some embodiments, there is provided a method of treatment, prevention or amelioration of psoriasis by topical administration to a subject in need thereof of a composition comprising a therapeutically effective dose of tapinarof, a therapeutically effective dose of at least one superpotent or potent corticosteroid (Class 1-2) and a carrier suitable for topical administration.

In some other embodiments, there is provided a method of treatment, prevention or amelioration of psoriasis by topical administration to a subject in need thereof of a composition comprising a therapeutically effective dose of tapinarof in combination with a therapeutically effective dose of at least one superpotent or potent corticosteroid (Class 1-2), a therapeutically effective dose of a JAK inhibitor and a carrier suitable for topical administration.

Treatment of Atopic Dermatitis (AD) with Tapinarof Combinations

According to some embodiments, there is provided a method of treatment, prevention or amelioration of atopic dermatitis, comprising the administration of a therapeutically effective dose of tapinarof, a therapeutically effective dose of at least one additional active agent selected from of at least one medium or low potency corticosteroid (Class 4-7), at least one JAK inhibitor, combinations thereof and a carrier suitable for topical administration.

According to some embodiments, there is provided a method of treatment, prevention or amelioration of atopic dermatitis, comprising the administration between 0.25-5% w/w or between 0.25-2% w/w tapinarof, a therapeutically effective dose of at least one additional active agent selected from of at least one medium or low potency corticosteroid (Class 4-7), at least one JAK inhibitor, combinations thereof and a carrier suitable for topical administration.

According to some embodiments, there is provided a method of treatment, prevention or amelioration of atopic dermatitis, comprising the administration of a therapeutically effective dose of tapinarof, a therapeutically effective dose of at least one medium or low potency corticosteroid (Class 4-7) and a carrier suitable for topical administration.

According to some embodiments, there is provided a method of treatment, prevention or amelioration of atopic dermatitis, comprising the administration between 0.25-5% w/w or 0.25-2% w/w tapinarof, a therapeutically effective dose of at least one medium or low potency corticosteroid (Class 4-7) and a carrier suitable for topical administration.

According to some embodiments, there is provided a method of treatment, prevention or amelioration of atopic dermatitis, comprising the administration of a therapeutically effective dose of tapinarof, a therapeutically effective dose of at least one JAK inhibitor and a carrier suitable for topical administration.

According to some embodiments, there is provided a method of treatment, prevention or amelioration of atopic dermatitis, comprising the administration between 0.25-5% w/w or 0.25-2% w/w tapinarof, a therapeutically effective dose of at least one medium or low potency corticosteroid (Class 4-7) and a carrier suitable for topical administration.

According to some embodiments, there is provided a method of treatment, prevention or amelioration of atopic dermatitis, comprising the administration of a therapeutically effective dose of tapinarof, a therapeutically effective dose of at least one medium or low potency corticosteroid (Class 4-7), a therapeutically effective dose of a JAK1 or a JAK2 inhibitor and a carrier suitable for topical administration.

According to some embodiments, there is provided a method of treatment, prevention or amelioration of atopic dermatitis, comprising the administration between 0.25-5% w/w or 0.25-2% w/w tapinarof, a therapeutically effective dose of at least one medium or low potency corticosteroid (Class 4-7), a therapeutically effective dose of a JAK1 or a JAK2 inhibitor and a carrier suitable for topical administration.

According to some embodiments, there is provided a method of treatment, prevention or amelioration of atopic dermatitis, comprising the administration of 1% w/w tapinarof, 0.1% w/w triamcinolone, 1.5% w/w ruxolitinib and a carrier suitable for topical administration.

According to some embodiments, there is provided a method of treatment of atopic dermatitis, comprising the administration of 0.5% w/w tapinarof, 0.05% w/w triamcinolone, 0.75% w/w ruxolitinib and a carrier suitable for topical administration.

The above composition may be a solution, suspension, emulsion or the like and is formulated as a cream, a gel, an ointment, an emulsion, a solution, an elixir, a lotion, a suspension, a tincture, a paste, a foam, an aerosol, a spray, a patch or any other formulation suitable for topical administration.

The above Class 4-7 corticosteroids are selected from the following steroids (strengths are indicated in parentheses).
Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).
Class 5—lower mid-strength, comprising 7 steroids: fluocinolone acetonide (0.01%), flurandrenolide (0.05%), fluticasone propionate (0.05%), prednicarbate (0.1%), desonide (0.05%), hydrocortisone (0.1%), hydrocortisone valerate (0.2%).
Class 6—mild, comprising only 3 steroids: alclomethasone dipropionate (0.05%), fluocinolone acetonide (0.01%), desonide (0.05%),
Class 7—least potent, comprising only one steroid: hydrocortisone (0.5%, 1%, 2%, 2.5%).

### Kits

Kits containing the combination compositions optionally including instructions for administration are provided. The combinations include, for example, the compositions as provided herein, optionally one or more reagents or solutions for diluting the compositions to a desired concentration for administration to a host subject, including human beings. Additionally, provided herein are kits containing the above-described combinations and optionally instructions for administration by topical, transdermal, or other routes, depending on the agent(s) to be delivered.

The compositions provided herein can be packaged as articles of manufacture containing packaging material, a composition provided herein, and a label that indicates that the composition is for treating a skin disorder, such psoriasis or atopic dermatitis, and is formulated for topical or transdermal delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

In some embodiments, there is provided a topical composition comprising from about 0.25% w/w to about 5.0% w/w tapinarof or from about 0.25% w/w to about 2.0% w/w tapinarof and at least one additional active agent, selected from about 0.01% w/w to about 0.25% w/w at least one corticosteroid of potency class 1 or 2 or from about 0.0025% w/w to about 2.5% w/w at least one corticosteroid of potency class 3-7, from about 0.001% w/w to about 0.005% w/w calcipotriene, from about 0.1% w/w to about 3% w/w at least one JAK1, JAK2 or JAK 1/2 inhibitor and combinations thereof, and a carrier suitable for topical administration.

In some other embodiments, there is provided a composition comprising from about 0.25% w/w to about 5.0% w/w tapinarof or from about 0.25% w/w to about 2.0% w/w tapinarof and at least one additional active agent selected from about 0.01% w/w to about 0.25% w/w at least one corticosteroid of potency class 1 or 2 and combinations thereof, and a carrier suitable for topical administration.

According to some embodiments, there is provided a composition comprising from about 0.25% w/w to about 5.0% w/w tapinarof or from about 0.25% w/w to about 2.0% w/w tapinarof and at least one additional active agent selected from about 0.0025% w/w to about 2.5% w/w at least one corticosteroid of potency class 3-7 and combinations thereof, and a carrier suitable for topical administration.

According to some other embodiments, there is provided a composition comprising from about 0.25% w/w to about 5.0% w/w tapinarof or from about 0.25% w/w to about 2.0% w/w tapinarof and at least one additional active agent selected from about 0.01% w/w to about 0.25% w/w at least one corticosteroid of potency class 1 or 2, from about 0.001% w/w to about 0.005% w/w calcipotriene and combinations thereof, and a carrier suitable for topical administration.

In some embodiments, there is provided a composition of comprising from about 0.25% w/w to about 5.0% w/w tapinarof or from about 0.25% w/w to about 2.0% w/w tapinarof and at least one additional active agent selected from about 0.0025% w/w to about 2.5% w/w at least one corticosteroid of potency class 3-7, from about 0.001% w/w to about 0.005% w/w calcipotriene and combinations thereof, and a carrier suitable for topical administration.

In some other embodiments, there is provided a composition comprising from about 0.25% w/w to about 5.0% w/w tapinarof or from about 0.25% w/w to about 2.0% w/w tapinarof and at least one additional active agent selected from about 0.1% w/w to about 3% w/w at least one JAK1, JAK2 or JAK 1/2 inhibitor and combinations thereof, and a carrier suitable for topical administration.

In some embodiments, there is provided a composition comprising from about 0.25% w/w to about 5.0% w/w tapinarof or from about 0.25% w/w to about 2.0% w/w tapinarof, from about 0.0025% w/w to about 2.5% w/w at least one corticosteroid of potency class 3-7, from about 0.1% w/w to about 3% w/w at least one JAK1, JAK2 or JAK 1/2 inhibitor, combinations thereof and a carrier suitable for topical administration.

In some other embodiments, there is provided a dosage form comprising any of the compositions of this invention, wherein formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

According to some embodiments, there is provided a method of treatment, prevention or alleviation of a skin disorder selected from plaque, guttate, inverse, flexural/inverse pustular or erythrodermic psoriasis and atopic dermatitis, by once daily or twice daily topical administration to a subject in need thereof of any one of the compositions of this invention, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

According to some other embodiments, there is provided a method of treatment, prevention or alleviation of a skin disorder selected from plaque, guttate, inverse, flexural/inverse pustular or erythrodermic psoriasis, by once daily or twice daily topical administration to a subject in need thereof a composition comprising from about 0.25% w/w to about 5.0% w/w tapinarof or from about 0.25% w/w to about 2.0% w/w tapinarof and at least one additional active agent selected from about 0.01% w/w to about 0.25% w/w at least one corticosteroid of potency class 1 or 2 and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

In some embodiments, there is provided a method of treatment, prevention or alleviation of a skin disorder selected from plaque, guttate, inverse, flexural/inverse, pustular or erythrodermic psoriasis, by once daily or twice daily topical administration to a subject in need thereof a composition comprising from about 0.25% w/w to about 5.0% w/w tapinarof or from 0.25% w/w to about 2.0% w/w tapinarof, from about 0.0025% w/w to about 2.5% w/w at least one corticosteroid of potency class 3-7 and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

In some other embodiments, there is provided a method of treatment, prevention or alleviation of a skin disorder selected from plaque, guttate, inverse, flexural/inverse, pustular or erythrodermic psoriasis, by once daily or twice daily topical administration to a subject in need thereof a composition comprising from about 0.25% w/w to about 5.0% w/w tapinarof or from about 0.25% w/w to about 2.0% w/w tapinarof, from about 0.01% w/w to about 0.25% w/w at least one corticosteroid of potency class 1 or 2, from about 0.001% w/w to about 0.005% w/w calcipotriene and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

According to some embodiments, there is provided a method of treatment, prevention or alleviation of a skin disorder selected from plaque, guttate, inverse, flexural/inverse, pustular or erythrodermic psoriasis, by once daily or twice daily topical administration to a subject in need thereof a composition comprising from about 0.25% w/w to about 5.0% w/w tapinarof or from about 0.25% w/w to about 2.0% w/w tapinarof, from about 0.0025% w/w to about 2.5% w/w at least one corticosteroid of potency class 3-7, from about 0.001% w/w to about 0.005% w/w calcipotriene and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

According to some other embodiments, there is provided a method of treatment, prevention or alleviation of atopic dermatitis, by once daily or twice daily topical administration to a subject in need thereof a composition comprising from about 0.25% w/w to about 5.0% w/w tapinarof or from about 0.25% w/w to about 2.0% w/w tapinarof, from about 0.0025% w/w to about 2.5% w/w at least one corticosteroid of potency class 3-7, combinations thereof, and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

In some embodiments, there is provided a method of treatment, prevention or alleviation of atopic dermatitis, by once daily or twice daily topical administration to a subject in need thereof a composition comprising from about 0.25% w/w to about 5.0% w/w tapinarof or from about 0.25% w/w to about 2.0% w/w tapinarof, from about 0.1% w/w to about 3% w/w at least one JAK1, JAK2 or JAK 1/2 inhibitor and combinations thereof, and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

In some other embodiments, there is provided a method of treatment, prevention or alleviation of atopic dermatitis, by once daily or twice daily topical administration to a subject in need thereof of a composition comprising from about 0.25% w/w to about 5.0% w/w tapinarof or from about 0.25% w/w to about 2.0% w/w tapinarof, from about 0.0025% w/w to about 2.5% w/w at least one corticosteroid of potency class 3-7, from about 0.1% w/w to about 3% w/w at least one JAK1, JAK2 or JAK 1/2 inhibitor and combinations thereof, and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

According to some embodiments, there is provided a regimen of administration comprising the once daily or twice daily administration to a patient in need thereof of a dosage form comprising any one of the compositions of this invention, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch or an applicator syringe, until remission or according to doctor's orders.

According to some other embodiments, there is provided a kit comprising at least one dosage form comprising any one of the compositions of this invention, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch or an applicator syringe, and instructions for use.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “calcipotriene” as used herein refers to calcipotriene or to hydrate thereof.

As used herein, the term “treating” or” treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

As used herein, the term “essentially free” generally refers to a composition having less than about 2 percent by weight, more preferably 1 percent per weight, less than about 0.5 percent by weight or even less than 0.1 percent by weight of a certain ingredient, based on the total weight of the composition.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

### Example 1

Preparation of a Tapinarof/Betamethasone Dipropionate Cream Composition for the Treatment of Psoriasis

The topical tapinarof/betamethasone dipropionate combination cream consists of:
0.25-2.0% w/w tapinarof,
0.01-0.05% w/w betamethasone dipropionate,
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
6.0-7.0% of 0.1M NaOH or HCl as an aqueous phase pH,
Make up to 100% with purified water and
Adjust the pH to 6.0-7.0 with 0.1 M NaOH or HCl

The cream composition is prepared by the following steps:
(1) weigh tapinarof having an average particle size of less than 1 μm;
(2) heat the propylene glycol to 60° C. in a water bath;
(3) add to the heated propylene glycol while stirring tapinarof, betamethasone dipropionate, BHT, menthol, octadecanol, polysorbate 80 and glyceryl monostearate, and dissolve to obtain an oil phase;
(4) prepare the aqueous phase by heating purified water in a water bath to 60° C., stir in and dissolve polysorbate 80 and adjust the pH to 6.0-7.0 with 0.1 M NaOH or HCl;
(5) add the aqueous phase to the oil phase under vacuum stirring, and cool to room temperature to obtain a cream;
(6) fill the tapinarof/betamethasone dipropionate combination cream in an aluminum tube or other delivery system.

### Example 2

Preparation of a Tapinarof/Calcipotriene Cream Composition for the Treatment of Psoriasis

The topical tapinarof/calcipotriene combination cream consists of:
0.25-2.0% w/w tapinarof,
0.001-0.005% w/w calcipotriene or calcipotriene hydrate (calculated as calcipotriene),
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
6.0-7.0% of 0.1M NaOH or HCl as an aqueous phase pH,
Make up to 100% with purified water and
Adjust the pH to 6.0-7.0 with 0.1 M NaOH or HCl

The cream composition is prepared by the following steps:
(1) weigh tapinarof having an average particle size of less than 1 μm;
(2) heat the propylene glycol to 60° C. in a water bath;
(3) add to the heated propylene glycol while stirring tapinarof, calcipotriene, BHT, menthol, octadecanol, polysorbate 80 and glyceryl monostearate, and dissolve to obtain an oil phase;
(4) prepare the aqueous phase by heating purified water in a water bath to 60° C., stir in and dissolve polysorbate 80 and adjust the pH to 6.0-7.0 with 0.1 M NaOH or HCl;
(5) add the aqueous phase to the oil phase under vacuum stirring, and cool to room temperature to obtain a cream;
(6) fill the tapinarof/calcipotriene combination cream in an aluminum tube or other delivery system.

### Example 3

Preparation of a Tapinarof/Calcipotriene/Betamethasone Dipropionate Cream Composition for the Treatment of Psoriasis

The topical tapinarof/calcipotriene/betamethasone dipropionate combination cream consists of:
0.25-2.0% w/w tapinarof,
0.001-0.005% w/w calcipotriene or calcipotriene hydrate (calculated as calcipotriene),
0.01-0.05% w/w betamethasone dipropionate,
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
6.0-7.0% of 0.1M NaOH or HCl as an aqueous phase pH,
Make up to 100% with purified water and
Adjust the pH to 6.0-7.0 with 0.1 M NaOH or HCl

The cream composition is prepared by the following steps:
(1) weigh tapinarof having an average particle size of less than 1 μm;
(2) heat the propylene glycol to 60° C. in a water bath;
(3) add to the heated propylene glycol while stirring tapinarof, calcipotriene, betamethasone dipropionate, BHT, menthol, octadecanol, polysorbate 80 and glyceryl monostearate, and dissolve to obtain an oil phase;
(4) prepare the aqueous phase by heating purified water in a water bath to 60° C., stir in and dissolve polysorbate 80 and adjust the pH to 6.0-7.0 with 0.1 M NaOH or HCl;
(5) add the aqueous phase to the oil phase under vacuum stirring, and cool to room temperature to obtain a cream;
(6) fill the tapinarof/calcipotriene/betamethasone dipropionate combination cream in an aluminum tube or other delivery system.

### Example 4

Preparation of a Tapinarof/Triamcinolone Cream Composition for the Treatment of Atopic Dermatitis

The topical tapinarof/triamcinolone combination cream consists of:
0.25-2.0% w/w tapinarof,
0.05-0.2% w/w triamcinolone,
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
6.0-7.0% of 0.1M NaOH or HCl for adjusting the pH to 6.0-7.0
Purified water up to 100%

The cream composition is prepared by the following steps:
(1) weigh tapinarof having an average particle size of less than 1 μm;
(2) heat the propylene glycol to 60° C. in a water bath;
(3) add to the heated propylene glycol while stirring tapinarof, triamcinolone, BHT, menthol, octadecanol, polysorbate 80 and glyceryl monostearate, and dissolve to obtain an oil phase;
(4) prepare the aqueous phase by heating purified water in a water bath to 60° C., stir in and dissolve polysorbate 80 and adjust the pH to 6.0-7.0 with 0.1 M NaOH or HCl;
(5) add the aqueous phase to the oil phase under vacuum stirring, and cool to room temperature to obtain a cream;
(6) fill the tapinarof/triamcinolone combination cream in an aluminum tube or other delivery system.

### Example 5

Preparation of a Tapinarof/Ruxolitinib Cream Composition for the Treatment of Atopic Dermatitis

The topical tapinarof/ruxolitinib combination cream consists of:
0.25-2.0% w/w tapinarof,
1-2% w/w ruxolitinib phosphate (calculated as base),
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
6.0-7.0% of 0.1M NaOH or HCl for adjusting the pH to 6.0-7.0
Purified water up to 100%

The cream composition is prepared by the following steps:
(1) weigh tapinarof having an average particle size of less than 1 μm;
(2) heat the propylene glycol to 60° C. in a water bath;
(3) add to the heated propylene glycol while stirring tapinarof, ruxolitinib phosphate, BHT, menthol, octadecanol, polysorbate 80 and glyceryl monostearate, and dissolve to obtain an oil phase;
(4) prepare the aqueous phase by heating purified water in a water bath to 60° C., stir in and dissolve polysorbate 80 and adjust the pH to 6.0-7.0 with 0.1 M NaOH or HCl;
(5) add the aqueous phase to the oil phase under vacuum stirring, and cool to room temperature to obtain a cream;
(6) fill the tapinarof/ruxolitinib combination cream in an aluminum tube or other delivery system.

### Example 6

Preparation of a Tapinarof/Triamcinolone/Ruxolitinib Cream Composition for the Treatment of Atopic Dermatitis

The topical tapinarof/triamcinolone/ruxolitinib combination cream consists of:
0.25-2.0% w/w tapinarof,
0.05-0.2% w/w triamcinolone,
1-2% w/w ruxolitinib phosphate (calculated as base),
0.1-0.5% w/w menthol,
0.01-0.05% w/w butylated hydroxyanisole (BHA),
15-30% w/w propylene glycol,
5.0-15.0% polysorbate 80,
10-25% w/w glyceryl monostearate,
10-25% w/w of thickener octadecanol,
6.0-7.0% of 0.1M NaOH or HCl for adjusting the pH to 6.0-7.0
Purified water up to 100%

The cream composition is prepared by the following steps:
(1) weigh tapinarof having an average particle size of less than 1 μm;
(2) heat the propylene glycol to 60° C. in a water bath;
(3) add to the heated propylene glycol while stirring tapinarof, triamcinolone, ruxolitinib phosphate, BHT, menthol, octadecanol, polysorbate 80 and glyceryl monostearate, and dissolve to obtain an oil phase;
(4) prepare the aqueous phase by heating purified water in a water bath to 60° C., stir in and dissolve polysorbate 80 and adjust the pH to 6.0-7.0 with 0.1 M NaOH or HCl;
(5) add the aqueous phase to the oil phase under vacuum stirring, and cool to room temperature to obtain a cream;
(6) fill the tapinarof/triamcinolone/ruxolitinib combination cream in an aluminum tube or other delivery system.

### Example 7

Preparation of Tapinarof, 1% Lotion

TABLE 1
The topical tapinarof lotion consisted of:

| Raw Material (compendial Name) | % w/w |
|---|---|
| Tapinarof 98% | 1.0 |
| Castor oil | 4.0 |
| Mineral oil light | 4.0 |
| Diethylene glycol monoethyl ether | 5.5 |
| Dimethyl Sulfoxide | 5.5 |
| Sorbitan Monooleate | 0.1 |
| Propylene glycol | 10.0 |
| Disodium Edetate (EDTA) | 0.1 |
| Carbomer Copolymer Type B Pemulen ®TR-1 | 0.4 |
| Carbomer Homopolymer Type A Carbopol ®981 | 0.6 |
| Purified water | 64.00 |
| Benzoic Acid | 0.25 |
| BHT | 0.1 |
| Citric Acid | 0.1 |
| Sodium Citrate | 0.2 |
| Sodium hydroxide pellets | For pH adjustment |
| Purified water | q.s. to 100 |

### Water Phase

Into a glass beaker water and Benzoic Acid were added. The beaker was placed inside a hot water bath adjusted to 60° C. and the mixture was mixed with a magnetic stirrer until a clear solution free from particles was obtained. Then EDTA, Citric Acid and Sodium Citrate were added. The mixing was continued until a clear solution was obtained. The solution was cooled down to room temperature. Then, the pH was slowly adjusted to pH 6.0 with NaOH 20%.

### Oil Phase

In a separate glass beaker Mineral oil light, castor oil, span 80 and BHT were weighed. The beaker was placed inside a hot water bath adjusted to 60° C. and the mixture was mixed with a magnetic stirrer until a uniform solution was obtained. Then Carbopol®981 and Pemulen®TR-1 were slowly added and the mixing was continued until a homogenous mixture was obtained. The mixture was cooled down to room temperature.

### Active Phase

Into a separate glass beaker Propylene Glycol, Transcutol and DMSO were weighed. The mixture was mixed with a magnetic stirrer until a uniform homogenous solution was obtained. The beaker was covered with an aluminum foil and placed in a yellow light hood. Tapinarof was slowly added, and the mixing was continued for about 1 h until a clear solution free from particles was obtained.

The oil phase was slowly added to the water phase while homogenizing for about 5 minutes, until there were no lumps. Then, the active phase was slowly added to the Water+Oil phase while homogenizing for about 5 minutes.

Water was added for batch completion, and final pH was measured to conform it is around pH 5.

### Example 8

Preparation of a Tapinarof Cream Composition

TABLE 2
The topical tapinarof cream consisted of:

| Material | % w/w | Function |
|---|---|---|
| Tapinarof | 1.0 | Active |
| Propylene glycol | 15 | Solvent, humectant |
| Diethylene glycol monoethyl ether | 5 | Solvent |
| (Transcutol P) | | |
| Mineral oil light | 4.0 | Emollient |
| Petrolatum | 2.0 | Emollient |
| Castor oil | 4.0 | Solvent, emollient |
| Butylated hydroxytoluene (BHT) | 0.1 | Antioxidant |
| Carbopol 980 | 1.0 | Gelling agent |
| Pemulen TR1 | 0.4 | Viscosity increasing agent, |
| | | Surfactant |
| Methylparaben | 0.3 | Preservative |
| Phenoxyethanol | 0.5 | Preservative |
| EDTA (Ethylendiaminetetraacetate | 0.1 | Chelating agent |
| Disodium salt) | | |
| Sodium hydroxide 20% | 1.0 | pH agent |
| Water | To 100 | Continuous phase |

The Cream Composition is Prepared by the Following Steps:

### Oil Phase Preparation.

Transcutol and BHT were transferred to 70 C water bath and mixed with a magnetic stirrer until full dissolution of BHT. Tapinarof was then added, and the mixing continued until full dissolution of tapinarof. Petrolatum, mineral oil light and castor oil were added and mixed with a magnetic stirrer until homogeneous dispersion of Petrolatum was obtained.

### Aqueous Phase

Water, propylene glycol, EDTA, phenoxyethanol and methylparaben were mixed at 150±50 rpm and heated to 65° C. At 65±2° C., Carbopol 980 and pemulen TR1 were added under high shear homogenization for 10 min.

High shear homogenization was stopped and NaOH 20% was added, then additional stirring was applied at 60° C. The pH was then measured.

### Emulsion:

The oil phase was added to the aqueous phase under high shear homogenization at for 10 min at 60° C. Then the homogenization was stopped, and the emulsion was cooled to room temperature.

### Example 9

Tapinarof Cream, 1% with Docusate Sodium (=Dioctyl Sodium Sulfosuccinate) as surfactant

TABLE 3
Tapinarof composition with docusate sodium

| Material | % w/w | Function |
|---|---|---|
| Tapinarof | 1.0 | Active ingredient |
| Propylene glycol | 10.0 | Solvent, humectant |
| Diethylene glycol monoethyl ether | 10.0 | Solvent |
| (Transcutol) | | |
| Mineral oil light | 4.0 | Emollient |
| Petrolatum | 2.0 | Emollient |
| Castor oil | 4.0 | Solvent, Emollient |
| Butylated hydroxytoluene (BHT) | 0.1 | Antioxidant |
| Carbopol 980 | 1.0 | Gelling agent |
| Docusate sodium | 0.5 | Surfactant |
| Benzoic Acid | 0.1 | Preservative |
| EDTA (Ethylendiaminetetraacetate | 0.1 | Chelating agent |
| Disodium salt) | | |
| Sodium hydroxide 20% | 1.5 | pH agent |
| Water | To 100 | Continuous phase |

The Cream Composition is Prepared by the Following Steps:

### Oil Phase Preparation

Petrolatum, mineral oil light and castor oil were heated to 60 C and mixed with a magnetic stirrer until homogeneity was obtained.

### Active Phase Preparation

Transcutol and BHT were transferred to 60° C. water bath and mixed with a magnetic stirrer until full dissolution of BHT. Tapinarof was then added, and the mixing continued until full dissolution of tapinarof.

Aqueous Phase Water, propylene glycol, EDTA, benzoic acid and docusate sodium were mixed at 150±50 rpm and heated to 50° C. Carbopol 980 was added under high shear homogenization for 15 min.

High shear homogenization was stopped and NaOH 20% was added, then additional stirring was applied at 50° C. The pH was then measured.

### Emulsion:

The oil phase+active phase were added to the aqueous phase under high shear homogenization at 60° C. for 10 min. Then the homogenization was stopped, and the emulsion was cooled to room temperature.

TABLE 4
Stability results of the tapinarof composition with docusate sodium:
Tapinarof cream, 1%

| Test | T = 0 | 2 weeks 40° C. | 1.5 months 40° C. |
|---|---|---|---|
| Assay | 101.9 | 102.1 | 102.3 |

### Example 10

Tapinarof Cream, 1% with Sodium Lauryl Sulfate as Surfactant

TABLE 5
Tapinarof composition with sodium lauryl sulfate

| Material | % w/w | Function |
|---|---|---|
| Tapinarof | 1.0 | Active ingredient |
| Propylene glycol | 10.0 | Solvent, humectant |
| Diethylene glycol monoethyl ether | 10.0 | Solvent |
| (Transcutol) | | |
| Mineral oil light | 4.0 | Emollient |
| Petrolatum | 2.0 | Emollient |
| Castor oil | 4.0 | Solvent, emollient |
| Butylated hydroxytoluene (BHT) | 0.1 | Antioxidant |
| Carbopol 980 | 1.0 | Gelling agent |
| Sodium lauryl sulfate | 0.5 | Surfactant |
| Benzoic Acid | 0.1 | Preservative |
| EDTA (Ethylendiaminetetraacetate | 0.1 | Chelating agent |
| Disodium salt) | | |
| Sodium hydroxide 20% | 1.5 | pH agent |
| Water | To 100 | Continuous phase |

The Cream Composition is Prepared by the Following Steps:

### Active Phase Preparation

Transcutol and BHT were transferred to 60° C. water bath and mixed with a magnetic stirrer until full dissolution of BHT. Tapinarof was then added, and the mixing continued until full dissolution of tapinarof.

### Aqueous Phase

Water, propylene glycol, EDTA, benzoic acid and sodium lauryl sulfate were mixed at 150±50 rpm and heated to 50° C. Carbopol 980 was added under high shear homogenization for 15 min.

High shear homogenization was stopped and NaOH 20% was added, then additional stirring was applied at 50° C. The pH was then measured.

### Emulsion:

The oil phase+active phase were added to the aqueous phase under high shear homogenization at for 10 min at 60° C. Then the homogenization was stopped, and the emulsion was cooled to room temperature.

### Example 11

Tapinarof Cream, 1% with PEG 100 Stearate (Non-Ionic) as Surfactant

TABLE 6
Tapinarof composition with PEG 100 stearate (non-ionic)

| Material | % w/w | Function |
|---|---|---|
| Tapinarof | 1.0 | Active ingredient |
| Propylene glycol | 10.0 | Solvent, humectant |
| Diethylene glycol monoethyl ether | 10.0 | Solvent |
| (Transcutol) | | |
| Mineral oil light | 4.0 | Emollient |
| Petrolatum | 2.0 | , emollient |
| Castor oil | 4.0 | Solvent, emollient |
| Butylated hydroxytoluene (BHT) | 0.1 | Antioxidant |
| Carbopol 980 | 1.0 | Gelling agent |
| PEG 100 stearate | 0.8 | Surfactant |
| Benzoic Acid | 0.1 | Preservative |
| EDTA (Ethylendiaminetetraacetate | 0.1 | Chelating agent |
| Disodium salt) | | |
| Sodium hydroxide 20% | 1.5 | pH agent |
| Water | To 100 | Continuous phase |

The Cream Composition is Prepared by the Following Steps:

### Oil Phase Preparation

Petrolatum, mineral oil light, PEG 100 stearate and castor oil were heated to 60 C and mixed with a magnetic stirrer until homogeneity was obtained.

### Active Phase Preparation

Transcutol and BHT were transferred to 60 C water bath and mixed with a magnetic stirrer until full dissolution of BHT. Tapinarof was then added, and the mixing continued until full dissolution of tapinarof.

### Aqueous Phase

Water, propylene glycol, EDTA, and benzoic acid were mixed at 150±50 rpm and heated to 50° C. Carbopol 980 was added under high shear homogenization for 15 min.

High shear homogenization was stopped and NaOH 20% was added, then additional stirring was applied at 50° C. The pH was then measured.

### Emulsion:

The oil phase+active phase were added to the aqueous phase under high shear homogenization at for 10 min at 60° C. Then the homogenization was stopped, and the emulsion was cooled to room temperature.

### Example 12

Psoriasis Treatment Using Tapinarof Compositions

### Goal

The objective of this study was to determine the efficacy of tapinarof composition as described in Example 8 compared to the vehicle and Dexamethasone as shown below. The evaluation was based on histology analysis following application of the respective test formulations.

This study is focused on the effect of tapinarof on experimentally induced psoriasis. In this model, a psoriasis phenotype is induced in normal skin grafted onto beige-severe combined immunodeficient mice by intradermal injection of natural killer/T-cells derived from psoriatic patients (Gilhar et al., Psoriasis is mediated by a cutaneous defect triggered by activated immunocytes: induction of psoriasis by cells with natural killer receptors, J. Invest. Dermatol. 2002 August; 119(2):384-91).

### Experimental Details

In order to address the aim of the experiment, the psoriatic humanized mice were established Additionally, 10 psoriatic patients were recruited (six males and four females), ranging from 21 to 66 years (mean age 45 years). All patients had classic plaque psoriasis. Normal skin from one healthy volunteer was obtained for grafting.

The efficacy study has been conducted on female mice. The total duration of the experiment was 8 weeks, upon receiving of the human skin. Normal skin from healthy volunteers were obtained for grafting. Healthy human abdominal skin pieces with a width of 0.4 mm and surface area of 1.5×1.5 cm were provided. The skin sample was preserved in isotonic saline solution and transplanted within 6-12 hours after skin donation. In addition, blood samples were collected from psoriatic patients having classic plaque psoriasis and not undergoing any treatment. 20 mL blood samples were taken from both males and females psoriatic patients (from psoriatic patients having classic plaque psoriasis and not undergoing any treatment), and peripheral blood mononuclear cells (PBMC) were separated immediately after blood withdrawal.

Mice: Beige-severe combined immunodeficient mice C.B-17/IcrHsdscid-bg (beige-SCID) mice (weight ˜25 g) were included in this study. Mice were monitored twice a week for vital signs such as weight, food intake, coat, eyes, anal genital areas, discharge/soiling, behavior and criteria related to skin transplantation. Normal healthy human donor skin were transplanted onto the beige-SCID mice as previously described (See Keren A. et al., Novel nanosome delivery system combined with siRNA targeting the antimicrobial gene DFB4: a new approach for psoriasis management? ExpDermatol. 2014; 23:464-465; Bracke S. et a/., Targeted silencing of DEFB4 in a bioengineered skin-humanized mouse model for psoriasis: development of siRNA SECosome-based novel therapies. ExpDermatol. 2014; 23:199-201; Zaretsky M. et al., Directed evolution of a soluble human IL-17A receptor for the inhibition of psoriasis plaque formation in a mouse model. Chem Biol. 2013 21; 20:202-2; Gilhar A. et al., The beneficial effect of blocking Kv1.3 in the psoriasiform SCID mouse model. J Invest Dermatol. 2011; 131:118 124; Keren A. et al., Innate lymphoid cells 3 induce psoriasis in xenotransplanted healthy human skin. J Allergy Clin Immunol. 2018; 142:305-308.e6).

PBMC's from the psoriatic patient's blood were isolated and cultured in the presence of a high dose of IL-2; Prospec, 100 U/mL of media-RPMI 1640,10% human AB serum (Sigma, St. Louis, MO), 1% glutamine, 1% antibiotics (media components; BiologicalIndustries, Kibbutz Beit Haemeck, Israel), as previously described (Gilhar A. et al., The beneficial effect of blocking Kv1.3 in the psoriasiform SCID mouse model. J Invest Dermatol. 2011; 131:118-124; Nousbeck J. et al., IGFBP7 as a potential therapeutic target in Psoriasis. J Invest Dermatol. 2011; 131:1767-1770; Schafer PH. et al., Apremilast, a cAMP phosphodiesterase-4 inhibitor, demonstrates anti-inflammatory activity in vitro and in a model of psoriasis. British Journal of Pharmacology 2010; 159:842-855).

Four weeks following the engraftment, each mouse was injected (intradermally) with 1×10⁷ IL-2 enriched allogeneic PBMC's from psoriatic patients (1×10⁷ cells injected/mouse). Cells from different psoriasis patients were equally distributed between treatment groups. Each patient was represented in each treatment group.

Fourteen days following cells injections, the mice were divided into treatment groups (Table 1, see below) and treated according to the timeline indicated below (see Scheme 1). Each transplant was equally topically treated (50 μg) by using a sterile spatula.

Dexamethasone (D2915, Sigma): was freshly prepared for each administration by dilution in saline to final concentration (50 mg/ml). Dexamethasone serves as a positive control and was administered topically twice daily for 14 days following cells injections (at Day 42 following human skin transplantation) until the end of the study (Day 56).

The vehicle was administered topically (50 μg) once daily for 14 days.

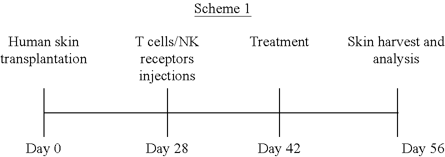

On Day 56, the entire skin graft was excised and placed in 10% formaldehyde in saline overnight. Then, the specimens were placed in 70% ethanol and embedded and stained according to the standard Hematoxylin/Eosin protocol.

Skin graft histological assessment was performed (according to the psoriatic criteria including epidermal thickness) using light microscopy, and two observers blindly performed the evaluations. Epidermal thickness was determined using an ocular micrometer at a minimum of 50 points along the epidermis selected to represent points of maximal and minimal thickness. Thickness of the suprapapillary plate was measured similarly at 50 points for each sample. Three slices were examined for each sample.

The experiment (study 1) comprised 30 mice divided into 3 groups (Table 1; with results found in Tables 7-9) as follows: Group 1: Vehicle; Group 2: Dexamethasone; Group 3: Tapinarof 1%, cream A (cream of Example 8).

Another experiment (study 2) comprised 30 mice divided into 3 groups (Table 11; with results found in Tables 12-15) as follows: Group 1: dexamethasone; Group 2: Tapinarof 1%ⁱ, cream D (Table 16); Group 3: Tapinarof 1%, cream A (cream of Example 8);

### Results

Evaluation of Normal Human Skin Grafts from Psoriatic T-Cell-Injected Beige-SCID Mice Study 1-Results

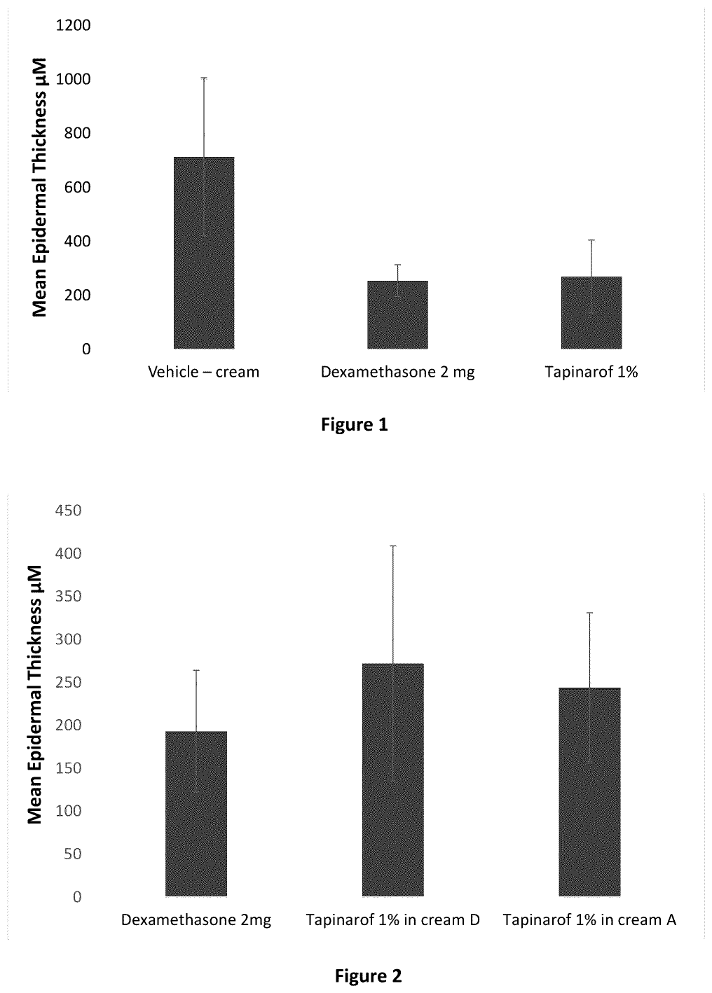

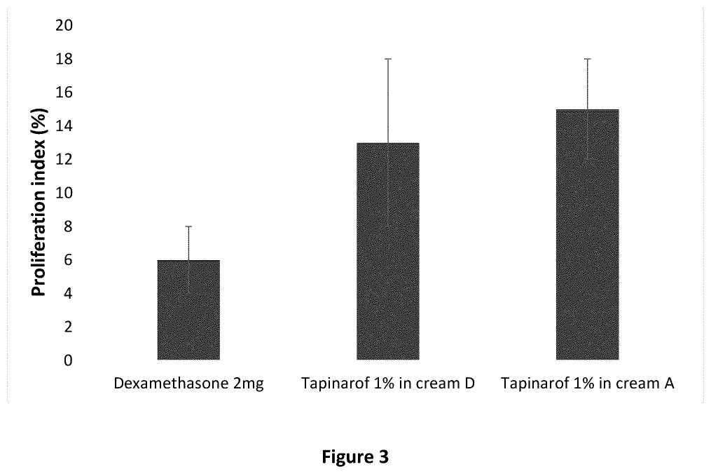

Normal human skin xenotransplanted onto beige-SCID mice treated with psoriatic T-cells expressing NK receptors, expressed psoriatic features that included epidermal thickening (acanthosis), enhanced epidermal proliferation, hyperkeratosis, parakeratosis, along with a dermal lymphocytic infiltrate, some areas with retention, others lacking the granular layer. Elongation of rete ridges was also observed in most psoriatic skin grafts, and vascular dilatation associated with a perivascular lymphocytic infiltrate was noted in the papillary dermis. Conversely, normal skin grafts from mice treated with dexamethasone showed complete (9/10) recovery from the psoriatic features. Specifically, these dexamethasone-treated grafts underwent a complete normalization of the skin, including a significant decrease in epidermal thickness compared to the Vehicle cream group (254±59μm versus 713±293μm, P<0.002) (Tables 7-9, FIG. 1).

TABLE 7
Study groups

| Group | Compound | Route | Frequency |
|---|---|---|---|
| 1 | Vehicle - cream | Topical | Once daily |
| 2 | Dexamethasone 2 mg | Topical | Twice a day |
| 3 | Tapinarof 1% (Cream A) | Topical | Once daily |

TABLE 8
Histological evaluation of human skin grafts following treatment

| | | | | Psoriatic | Partial | Complete |
|---|---|---|---|---|---|---|
| Group | Compound | Route | Frequency | Features | Recovery | Recovery |
| 1 | Vehicle - cream | Topical | Once daily | 9/10 | 1/10 | 0/10 |
| | | | | (90%) | (10%) | (0%) |
| 2 | Dexamethasone 2 mg | Topical | Twice a day | 0/10 | 1/10 | 9/10 |
| | | | | (0%) | (10%) | (90%) |
| 3 | Tapinarof 1% | Topical | Once daily | 1/10 | 1/10 | 8/10 |
| | | | | (10%) | (10%) | (80%) |

TABLE 9
Histology of treated psoriasis grafts (mean epidermal
thickness, scoring, complete recovery/total No grafts)

| | | | Mean | Histological | |
|---|---|---|---|---|---|
| | | | Epidermal | Scoring | Complete |
| Group | Compound | Frequency | Thickness | (Baker system) | Recovery |
| 1 | Vehicle - cream | Once daily | 713 ± 293 | 9.05 ± 1.2 | 0/10 (0%) |
| 2 | Dexamethasone 2 mg | Twice a day | 254 ± 59 | 0.7 ± 1.9 | 9/10 (90%) |
| 3 | Tapinarof 1% | Once daily | 270 ± 135 | 1.45 ± 2.5 | 8/10 (80%) |

TABLE 10
Pathological scores based on Baker's scoring
system (A[IQR]) (Average, SD).*

| Histological criteria | | | Tapinarof |
|---|---|---|---|
| according to | Vehicle - | | (Cream A) |
| Baker's system | cream | Dexamethasone | (1%) |
| Epidermal thickening | 0.5 ± 0 | 0.05 ± 0.1 | 0.1 ± 0.2 |
| (acanthosis) | | | |
| Suprapapillary epidermal | 0.5 ± 0 | 0.05 ± 0.1 | 0.05 ± 0.1 |
| thinning | | | |
| Hyperkeratosis | 0.45 ± 0.1 | 0.05 ± 0.1 | 0.05 ± 0.1 |
| Parakeratosis | 0.9 ± 0.3 | 0.1 ± 0.3 | 0.1 ± 0.3 |
| Agranulosis - | 0.9 ± 0.2 | 0.1 ± 0.3 | 0.1 ± 0.3 |
| hypogranulosis | | | |
| **Munro micro abscess | 1.9 ± 0.3 | 0 ± 0 | 0.2 ± 0.6 |
| Regular/abnormal | 1.5 ± 0 | 0.1 ± 0.4 | 0.3 ± 0.6 |
| elongation of rete ridges | | | |
| ***Blood vessels | 0.5 ± 0 | 0.05 ± 0.1 | 0.05 ± 0.1 |
| (tortuosity, elongation) | | | |
| Mononuclear cells infiltrate | 1.9 ± 0.3 | 0.2 ± 0.6 | 0.5 ± 0.4 |
| in the papillary dermis | | | |
| Total Score | 9.05 ± 1.2 | 0.7 ± 1.9 | 1.45 ± 2.5 |

*Many of the psoriatic pathohistological components are missing in the Dexa and the tested treated transplants, therefore the results are spread out and the SD is large. In contrast, all the psoriatic parameters are present in the vehicle group, therefore the SD is small and thus the results are close to the mean.
**Appearance of neutrophils in the upper spinous layer or in the corneal cell layer.
***Parameters of pathological/inflammatory angiogenesis, edema and vascular enlargement, increased tortuosity and elongation in the dermal papilla. Vehicle versus all treated groups (p < 0.004, Kruskal-Wallis test, followed by the Mann-Whitney U test).

### Study 2-Results

Normal human skin xenotransplanted onto beige-SCID mice treated with psoriatic T-cells expressing NK receptors, expressed psoriatic features that included epidermal thickening (acanthosis), enhanced epidermal proliferation, hyperkeratosis, parakeratosis, along with a dermal lymphocytic infiltrate, some areas with retention, others lacking the granular layer. Elongation of rete ridges was also observed in most psoriatic skin grafts, and vascular dilatation associated with a perivascular lymphocytic infiltrate was noted in the papillary dermis.

The results demonstrated (FIGS. 7-8) a therapeutic effect of Tapinarof 1% of Cream A demonstrating similar histological score (including epidermal thickness, proliferation index, Baker's scoring, immunological markers) as Cream D. The creams demonstrated a complete normalization of the psoriatic phenotype in 8/10 grafts, including decreased epidermal thickness in both groups.

In conclusion, the study confirmed a therapeutic effect of Tapinarof 1% in cream A, demonstrating a similar histological score as cream D, in the humanized mouse model for psoriasis. However, the surfactant of cream A, i.e. pemulen TR1, is found at much lower concentration (0.4% w/w) compared to the surfactants of cream D (sum of concentrations of polysorbate 80, stearate 2 and stearate 20 is 4.4% w/w).

TABLE 11
Study groups

| Group | Compound | Route | Frequency |
|---|---|---|---|
| 1 | Dexamethasone 2 mg | Topical | Twice a day |
| 2 | Tapinarof 1% (Cream D) | Topical | Once daily |
| 3 | Tapinarof 1% (Cream A) | Topical | Once daily |

TABLE 12
Histological evaluation of human skin grafts following treatment

| | | | | Psoriatic | Partial | Complete |
|---|---|---|---|---|---|---|
| Group | Compound | Route | Frequency | Features | Recovery | Recovery |
| 1 | Dexamethasone 2 mg | Topical | Twice a day | 0/10 | 0/10 | 10/10 |
| 2 | Tapinarof | Topical | Once daily | 1/10 | 1/10 | 8/10 |
| | 1% (Cream D) | | | | | |
| 3 | Tapinarof | Topical | Once daily | 1/10 | 1/10 | 8/10 |
| | 1% (Cream A) | | | | | |

TABLE 13
Histology of treated psoriasis grafts (mean epidermal
thickness, scoring, complete recovery/total No grafts)

| | | | | | Histological | |
|---|---|---|---|---|---|---|
| | | | Mean | Proliferation | Scoring | |
| | | | epidermal | index | (Baker | Complete |
| Group | Compound | Frequency | thickness | (%) | system) | Recovery |
| 1 | Dexamethasone 2 mg | Twice a day | 193 ± 71 | 6 ± 2 | 0.25 ± 0.8 | 10/10 |
| 2 | Tapinarof | Once daily | 272 ± 137 | 13 ± 5 | 1.35 ± 3.1 | 8/10 |
| | 1% (Cream D) | | | | | |
| 3 | Tapinarof | Once daily | 244 ± 87 | 15 ± 3 | 1 ± 2.3 | 8/10 |
| | 1% (Cream A) | | | | | |

TABLE 14
Pathological scores based on Baker's scoring system (A[IQR]) (Average, SD).*

| Histological criteria | | Tapinarof 1% | Tapinarof 1% |
|---|---|---|---|
| according to Baker's system | Dexamethasone | Cream D | Cream A |
| Epidermal thickening (acanthosis) | 0.05 ± 0.1 | 0.05 ± 0.1 | 0.1 ± 0.2 |
| Suprapapillary epidermal thinning | 0.05 ± 0.1 | 0.1 ± 0.2 | 0.1 ± 0.2 |
| Hyperkeratosis | 0 ± 0 | 0.05 ± 0.1 | 0.05 ± 0.1 |
| Parakeratosis | 0 ± 0 | 0.1 ± 0.3 | 0.1 ± 0.3 |
| Agranulosis - hypogranulosis | 0.1 ± 0.3 | 0.2 ± 0.4 | 0.2 ± 0.4 |
| **Munro micro abscess | 0 ± 0 | 0.2 ± 0.6 | 0 ± 0 |
| Regular/abnormal elongation of rete ridges | 0 ± 0 | 0.2 ± 0.5 | 0.2 ± 0.5 |
| ***Blood vessels (tortuosity, elongation) | 0.05 ± 0.1 | 0.05 ± 0.1 | 0 ± 0 |
| Mononuclear cells infiltrate | 0 ± 0 | 0.4 ± 0.8 | 0.25 ± 0.6 |
| in the papillary dermis | | | |
| Total Score | 0.25 ± 0.8 | 1.35 ± 3.1 | 1 ± 2.3 |

*Many of the psoriatic pathohistological components are missing in the Dexamethasone and the tested treated transplants, therefore the results are spread out and the SD is large.
**Appearance of neutrophils in the upper spinous layer or in the corneal cell layer.
***Parameters of pathological/inflammatory angiogenesis, edema and vascular enlargement, increased tortuosity and elongation in the dermal papilla.

TABLE 15
Immunohistochemical markers for psoriasis in human xenotransplants

| | | | Psoriasin | | | | |
|---|---|---|---|---|---|---|---|
| | | HBD2 | (S100A7) | CD4 | CD8 | IL-17 | IL-22 |
| Group | Compound | (Integrated intensity, %) | | Cells per 0.66 mm² | | | |
| 1 | Dexamethasone 2 mg | 5.8 ± 0.9 | 6.6 ± 2.1 | 0.5 ± 0.8 | 0.6 ± 0.6 | 0.6 ± 0.9 | 0.4 ± 0.8 |
| 2 | Tapinarof | 14 ± 6.4 | 18.2 ± 5.2 | 2.4 ± 0.7 | 2.6 ± 0.9 | 3 ± 1.1 | 1.9 ± 0.6 |
| | 1% (Cream D) | | | | | | |
| 3 | Tapinarof | 12.4 ± 4.5^{>} | 12.9 ± 3.1^{>} | 2.8 ± 1^{>} | 2.7 ± 0.9^{>} | 3.3 ± 1.5^{>} | 1.5 ± 0.4^{>} |
| | 1% (Cream A) | | | | | | |

TABLE 16
cream D, which is formulation 21 of Tapinarof (1%
w/w) in Table 8 of U.S. Pat. No. 10,195,160:

% w/w

Aqueous Phase

| | |
|---|---|
| Purified water | 64.68 |
| Sodium citrate | 0.19 |
| Citric acid | 0.08 |
| Disodium EDTA | 0.1 |

Oil Phase

| | |
|---|---|
| tapinarof | 1.00 |
| Propylene glycol | 10.00 |
| Diethylene glycol monoethyl ether | 2.00 |
| BHT | 0.10 |
| Benzoic acid | 0.25 |
| Emulsifying wax, NF | 7.20 |
| Medium chain triglycerides | 10.00 |
| Polysorbate 80 | 1.50 |
| Steareth 2 | 1.80 |
| Steareth 20 | 1.10 |
| Total | 100.00 |

1. A topical cream composition, comprising from about 1% w/w to about 2% w/w tapinarof or a pharmaceutically acceptable salt thereof and less than 1% w/w surfactant.

2. The composition according to claim 1, wherein the surfactant is selected from the group consisting of Carbomer Copolymer Type B, polysorbate 80, sorbitan monooleate, polysorbate 20, PEG 100 stearate, polysorbate 60, sodium dodecylsulfate, sodium lauryl sulfate, cetearyl alcohol, dioctyl sodium sulfosuccinate (=docusate sodium), glyceryl oleate, glyceryl stearate, poloxamers and combination thereof.

3. The composition according to claim 1, wherein the surfactant is ionic or non-ionic.

4. The composition according to claim 1, further comprising from about 0.1% to about 4% w/w gelling agent.

5. The composition according to claim 1, further comprising from about 0.01% to about 1% w/w anti-oxidant.

6. A topical cream composition, comprising from about 0.25% w/w to about 10.0% w/w tapinarof or a pharmaceutically acceptable salt thereof, from about 0.1% w/w to about 20% surfactant, and optionally from about 0.01% to about 1% w/w an anti-oxidant, wherein the surfactant is ionic.

7. The composition according to claim 6, wherein the composition comprises 1% or 1.5% w/w tapinarof.

8. The composition according to claim 6, wherein the surfactant is anionic.

9. The composition according to claim 8, wherein the surfactant is selected from the group consisting of Carbomer Copolymer Type B, sodium lauryl sulfate, sodium dodecylsulfate and dioctyl sodium sulfosuccinate (docusate sodium).

10. The composition according to claim 6, wherein the surfactant is Carbomer Copolymer Type B or docusate.

11. The composition according to claim 6, wherein the concentration of the surfactant is 5% w/w or less than 5% w/w.

12. The composition of claim 11, wherein the concentration of the surfactant is from 0.2% to 0.8% w/w.

13. The composition according to claim 6, wherein the composition further comprises from about 0.1% w/w to about 4% w/w gelling agent.

14. The composition according to claim 13, further comprising an additional gelling agent.

15. The composition according to claim 4, further comprising an additional gelling agent

16. The composition of claim 14, wherein said gelling agent and said additional gelling agent are each selected independently from a carbomer, Natrosol hydroxyethylcellulose, starch, pectin, gelatin, agar, alginic acid and salts thereof, carboxymethylcellulose, hydroxypropylmethylcellulose and any combination thereof.

17. The composition of claim 15, wherein said gelling agent and said additional gelling agent are each selected independently from a carbomer, Natrosol hydroxyethylcellulose, starch, pectin, gelatin, agar, alginic acid and salts thereof, carboxymethylcellulose, hydroxypropylmethylcellulose and any combination thereof.

18. The composition of claim 16, wherein said gelling agent is Carbomer Homopolymer Type C in an amount of about 0.1% to about 4% w/w.

19. The composition of claim 17, wherein said gelling agent is Carbomer Homopolymer Type C in an amount of about 0.1% to about 4% w/w.

20. The composition according to claim 1, further comprising an additional surfactant.

21. The composition of claim 20, wherein said additional surfactant is selected from the group consisting of Carbomer Copolymer Type B, polysorbate 80, sorbitan monooleate, polysorbate 20, PEG 100 stearate, polysorbate 60, sodium dodecylsulfate, sodium lauryl sulfate, cetearyl alcohol, dioctyl sodium sulfosuccinate, glyceryl oleate, glyceryl stearate, poloxamers and combination thereof.

22. The composition according to claim 6, further comprising an additional surfactant.

23. The composition of claim 22, wherein said additional surfactant is selected from the group consisting of Carbomer Copolymer Type B, polysorbate 80, sorbitan monooleate, polysorbate 20, PEG 100 stearate, polysorbate 60, sodium dodecylsulfate, sodium lauryl sulfate, cetearyl alcohol, dioctyl sodium sulfosuccinate, glyceryl oleate, glyceryl stearate, poloxamers and combination thereof.

24. The composition according to claim 5, wherein said composition comprises from about 0.01% to about 0.2% w/w anti-oxidant.

25. The composition according to claim 5, wherein said anti-oxidant is selected from butylated hydroxy toluene, parabens, propyl gallate, ascorbic acid, flavones, flavanones, flavonols, stilbenoids, gallic acid, cinnamic acid, ellagic acid, salicylic acid, curcumin, eugenol, citric acid, tocopherol, vitamin E, tocopherol polyethylene glycol succinate and any combination thereof.

26. The composition according to claim 6, wherein said composition comprises from about 0.01% to about 0.2% w/w anti-oxidant.

27. The composition according to claim 6, wherein said anti-oxidant is selected from butylated hydroxy toluene, parabens, propyl gallate, ascorbic acid, flavones, flavanones, flavonols, stilbenoids, gallic acid, cinnamic acid, ellagic acid, salicylic acid, curcumin, eugenol, citric acid, tocopherol, vitamin E, tocopherol polyethylene glycol succinate and any combination thereof.

28. The composition according to claim 1, wherein the purity of said tapinarof is greater than 97%.

29. The composition according to claim 6, wherein the purity of said tapinarof is greater than 97%.

30. A method of treating a skin disorder in a patient in need thereof, said method comprises administering to said patient the topical cream composition according to claim 1, wherein said skin disorder is selected from psoriasis, toxic and allergic contact eczema, atopic eczema, seborrheic eczema, lichen simplex, sunburn, pruritus, alopecia areata, hypertrophic scars, discoid lupus erythematosus, follicular and extensive pyodermas, acne, rosacea, atopic dermatitis, prurigo nodularis and hidradenitis suppurativa.

31. A method of treating a skin disorder in a patient in need thereof, said method comprises administering to said patient the topical cream composition according to claim 6, wherein said skin disorder is selected from psoriasis, toxic and allergic contact eczema, atopic eczema, seborrheic eczema, lichen simplex, sunburn, pruritus, alopecia areata, hypertrophic scars, discoid lupus erythematosus, follicular and extensive pyodermas, acne, rosacea, atopic dermatitis, prurigo nodularis and hidradenitis suppurativa.
